(19) United States
(12) Patent Application Publication
(10) Pub. No.: US 2008/0032278 A1
(43) Pub. Date: Feb. 7, 2008

(54) Engineered Tubular Tissue Structures

(76) Inventor: Derek Leigh Jones, Cardiff (GB)

Correspondence Address: YOUNG & THOMPSON, 745 SOUTH 23RD STREET, 2ND FLOOR, ARLINGTON, VA, 22202, US

(21) Appl. No.: 11/579,598

(22) PCT Filed: May 9, 2005
(86) PCT No.: PCT/GB05/01740

§ 371 Date: Nov. 6, 2006

(30) Foreign Application Priority Data
May 7, 2004 (GB) 0410177.0

(51) Int. Cl. A01N 1/00; C12M 1/00; C12N 5/06

(52) U.S. Cl. 435/1.1; 435/307.1; 435/402

(57) ABSTRACT

A method and apparatus and associated components are described for producing a cellular tissue structure including a wall surrounding a space or passage. A suspension of cells is introduced into a culture vessel (70) of elongate tubular form defining an internal tissue formation surface. The vessel is rotated about a rotary axis to cause the cells in the suspension to move under the influence of centrifugal force to collect and form a layer on the tissue formation surface. The layer is then exposed to a tissue culture medium. The tissue formation surface is preferably resiliently compressive and a pulsed or other time-varying pressure is preferably applied during at least the maturation phase of formation of the cellular tissue structure. Various forms of apparatus are described to allow a large array of elongate culture vessels to be built and serviced in common or individually.

This invention relates to methods and apparatus for producing cellular tissue structures of generally tubular form and in particular, but not exclusively, to blood vessels, ureters, bladders and vascularised mini organs.

There are many medical conditions where replacement blood vessels or the like are needed, for example in patients suffering heart disease, diabetes etc.

There have been previous proposals for the synthetic or engineered construction of cellular tissue structures, for example by culturing a fibroblast sheet and then rolling into a multi-layer vessel, as described in WO 01/37884. In Nasseri et al. “Dynamic Rotational Seeding And Cell Culture System For Vascular Tube Formation” Tissue Engineering Vol. 9, no 2, 2003, pp 291-299, there is described a dynamic rotational seeding system in which scaffolding tubes are placed in culture vessels containing a cell suspension and rotated at low speed.

In Thompson et al. “A Novel Pulsatile, Laminar Flow Bioreactor For The Development Of Issue-Engineered Vascular Structures” Tissue Engineering Vol. 8 no 6, 2002, pp 1038-1088 there is described a proposal in which pulsatile flow is induced in a bioreactor system using a mechanical ventilator.

It is an aim of this invention to provide a method for engineering cellular tissue structures of generally tubular form to provide enhanced properties. We have found that by providing a system in which the vessel in which the cellular tissue is engineered is rotated at least initially to cause the cell suspension to form a layer under the influence of centrifugal force, and thereafter in which the layer is subjected to a time varying pressure results in a tissue with enhanced properties.

It is also an aim of this invention to provide an apparatus in which cellular tissue may be formed and thereafter undergo a maturation process without requiring removal from the apparatus.

We have discovered that the properties of the tissue-engineered structure may be further enhanced by subjecting the tissue culture vessel to different rates of rotation thereby subjecting the culture vessel to G forces high enough to cause creation of a well-packed tissue structure prior to development of the tissue.

### SUMMARY OF THE INVENTION

According to one aspect of this invention, there is provided a method of producing a cellular tissue structure comprising a wall surrounding a space or passage, which method comprises the steps of:—

(i) introducing into a vessel at least one suspension of cells, said vessel having therein an internal tissue formation surface;

(ii) rotating said vessel about a rotary axis to cause the cells in said suspension to move under the influence of centrifugal force to collect and form a layer on said tissue formation surface, and

(iii) exposing said layer to a tissue culture medium by passing said tissue culture medium through said vessel.

The cellular tissue structure may be of generally tubular form.

Where a structure is required which has first and second layers, this may be produced by introducing a first cell suspension containing cells of the first type into said vessel and, following step (iii), introducing a second cell suspension containing cells of a second type into said vessel, and repeating steps (ii) and (iii) with said second cell suspension.

Likewise, if the structure is to have three different layers a third cell suspension containing cells of a third type may be introduced into the vessel and steps (ii) and (iii) may be repeated with the third cell suspension.

Preferably, said method includes the step of applying a time-varying pressure to said layer, and this may be done during and/or after exposing said layer to said tissue culture medium.

In different arrangements, the at least one cell suspension may comprise a suspension including a plurality of different cell types.

Preferably, said tissue formation surface comprises the inner surface of a generally tubular tissue formation insert element which fits within said vessel.

Preferably, said insert element is made of a resiliently deformable material such that the inner radial dimension thereof varies in response to said time-varying pressure.

In one arrangement the insert element may be made of a closed cell foam material. A further tubular insert element of suitable material may be provided within the tissue formation insert and in this case the tissue formation surface will be the inner surface of said tubular insert element. The tubular insert element may, for example, comprise a layer of agarose gel to assist release of the formed cellular tissue structure at the completion of said method. A tissue scaffolding structure may be provided in said vessel and associated with said tissue formation surface.

In the above method, each cell suspension may be caused or allowed to become generally uniform after introduction into said vessel by rotating said vessel during, and/or immediately after, step (i). This may be achieved for example by rotating the vessel at a relatively low rotational speed of, say, between 5 and 15 rpm to cause the cell suspension to become generally uniform. In one arrangement, during at least an initial phase of step (ii) the vessel may be rotated at a relatively high rotational speed of say, between 500 and 3000 rpm. In a particular method the vessel may be rotated at a speed of between 1500 and 2500 rpm during an initial phase, with the speed thereafter being reduced to between 500 and 1500 rpm.

The step (ii) may be continued for a period of between 5 and 24 hours.

Said time-varying pressure may be a pulsed pressure cycle, and the frequency and/or magnitude of the pulses may increase with time. In a typical arrangement the magnitude of the pulses may lie in the range of from 1 gm/cm² to 200 gm/cm², and the frequency thereof may lie in the range from 10 pulses/minute to 80 pulses/minute.

In a particular method for e.g. the production of a blood vessel, the method may be carried out in two stages, during a first stage of which a tube is formed with three distinct cell layers, taking between 24 and 36 hours, and the second stage being the maturation of the tubular structure whilst under the influence of the pulse pressure system. This latter stage may take from 1 to 4 weeks, within the same apparatus.

According to another aspect of this invention there is provided apparatus for producing a cellular tissue structure comprising a wall surrounding a space or passage, said apparatus comprising:—
an elongate culture vessel having therein an internal tissue formation surface;
cell suspension supply means for introducing into said vessel at least one cell suspension;
mounting means for supporting said vessel for rotation about a rotary axis;
rotary drive means for rotating said vessel; and
pressure modulation means for varying the pressure within said vessel.

The invention also extends to an elongate culture vessel per se for use in the above apparatus.

Preferably said tissue formation surface comprises a radially inner surface of a tissue formation insert element removably located in said vessel. Said tissue formation insert element is preferably of elongate, generally tubular form. The tissue formation element may be made of a resiliently deformable material or include resiliently deformable portions. The tissue formation element may be made of a closed cell foam material. A further insert element of support material may be located within, or associated with, said tissue formation insert element, with the inner surface thereof defining the tissue formation surface. A tissue scaffolding structure may be associated with or located within said tissue formation surface.

The vessel may comprise a hollow cylindrical tube and respective end caps sealingly connected to opposite ends of said tube, each end cap including a flow passage communicating with the interior of said tube. One or both of the end caps may be removable. Each flow passage may be defined by a hollow tube removably connected to the associated end cap.

In another aspect, this invention provides apparatus for culturing cells, which apparatus comprises a plurality of generally elongate culture vessels mounted on a support means with their longitudinally axes generally parallel and each mounted for rotation about its respective longitudinal axis, and common drive means for rotating each of said culture vessels.

Whilst the invention has been described above, it extends to any inventive combination of the features set out above or in the following description.

Non-limiting embodiments of apparatus constructed in accordance with this invention will now be described, together with various non-limiting examples, for a better understanding of the invention, reference being made to the accompanying drawings in which:—

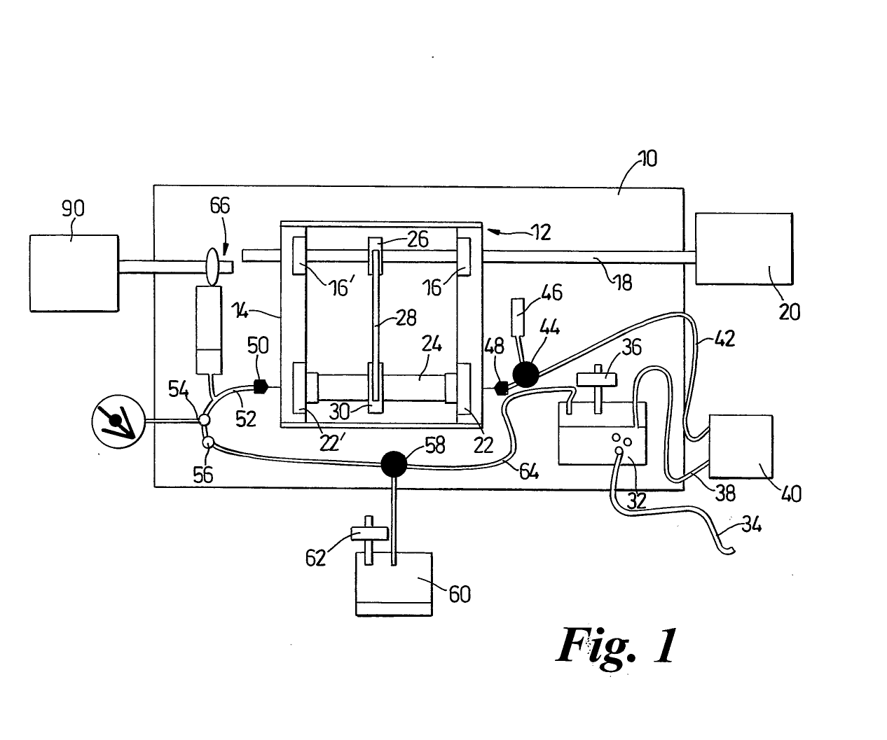

FIG. 1 is a schematic view of an apparatus in accordance with this invention;

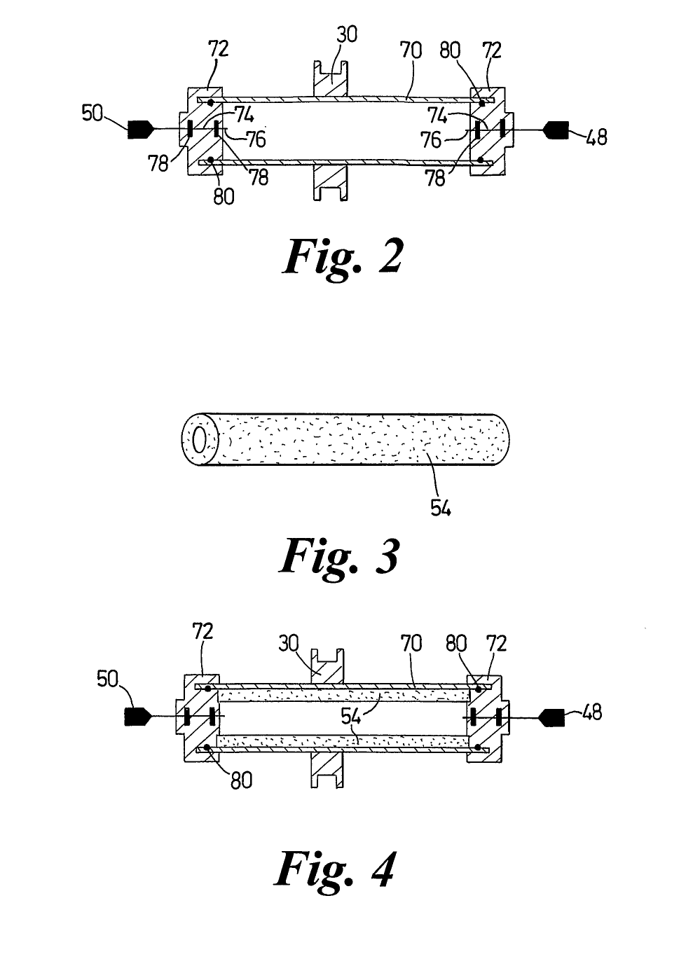

FIG. 2 is a more detailed view of a centrifugal tube assembly used in the apparatus of FIG. 1;

FIG. 3 is a view of a foam insert for insertion into the centrifugal tube assembly of FIG. 2;

FIG. 4 shows the centrifugal assembly of FIG. 2 but with the foam insert in situ;

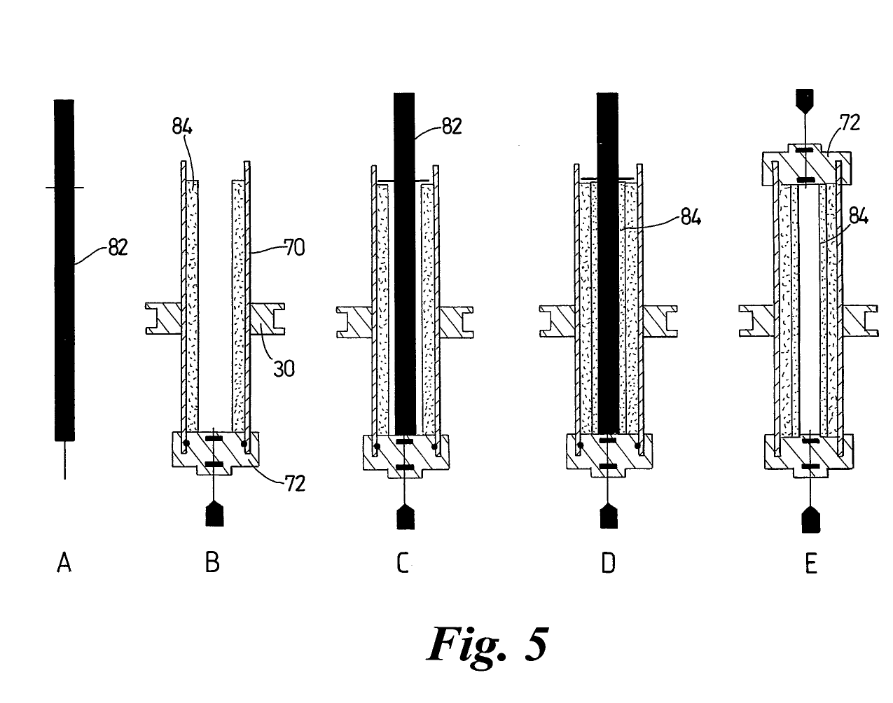

FIGS. 5(a) to (e) illustrate the formation of an agarose layer on the inner surface of the foam insert in the assembly of FIG. 4;

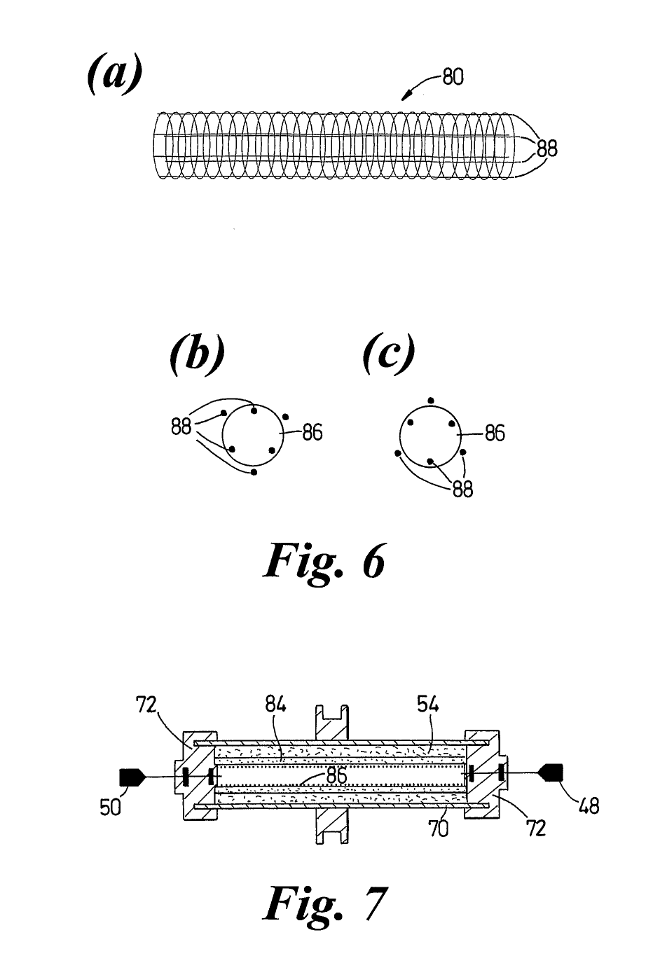

FIGS. 6(a) to (c) are side views and successive sectional views of a tissue scaffolding insert;

FIG. 7 is a view of the tissue scaffolding insert of FIG. 6 inserted into the assembly as illustrated in FIG. 5(e);

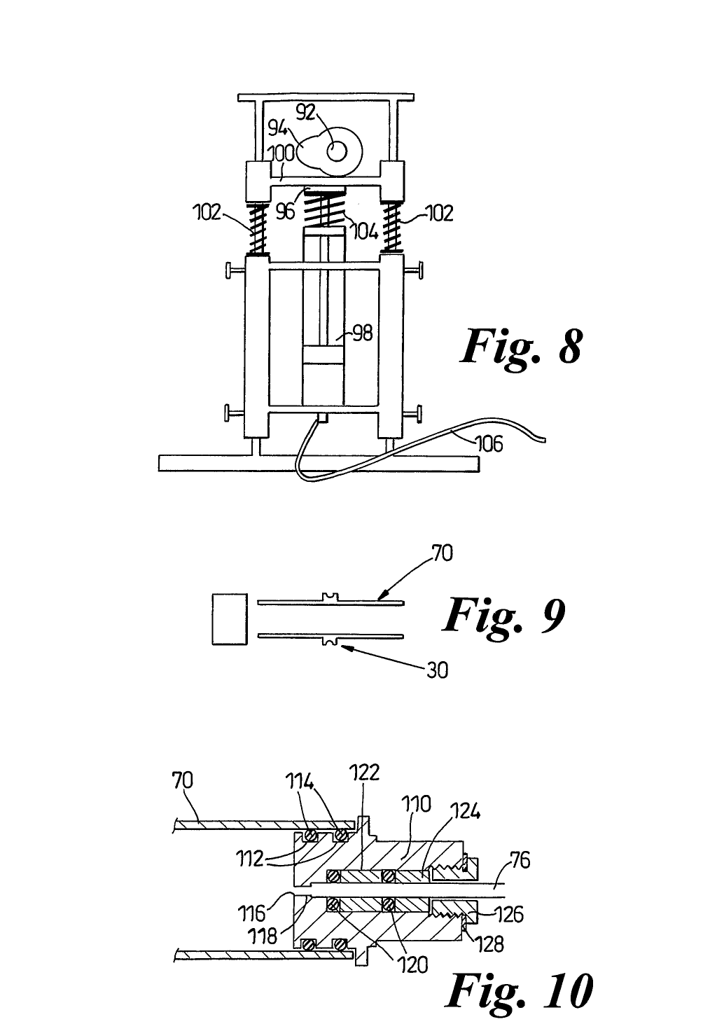

FIG. 8 is a view of the pressure pulse generator used in the apparatus of FIG. 1;

FIG. 9 is a schematic view illustrating a manufacturing step in producing a centrifugal tube for the apparatus;

FIG. 10 is a detailed view of one end cap assembly;

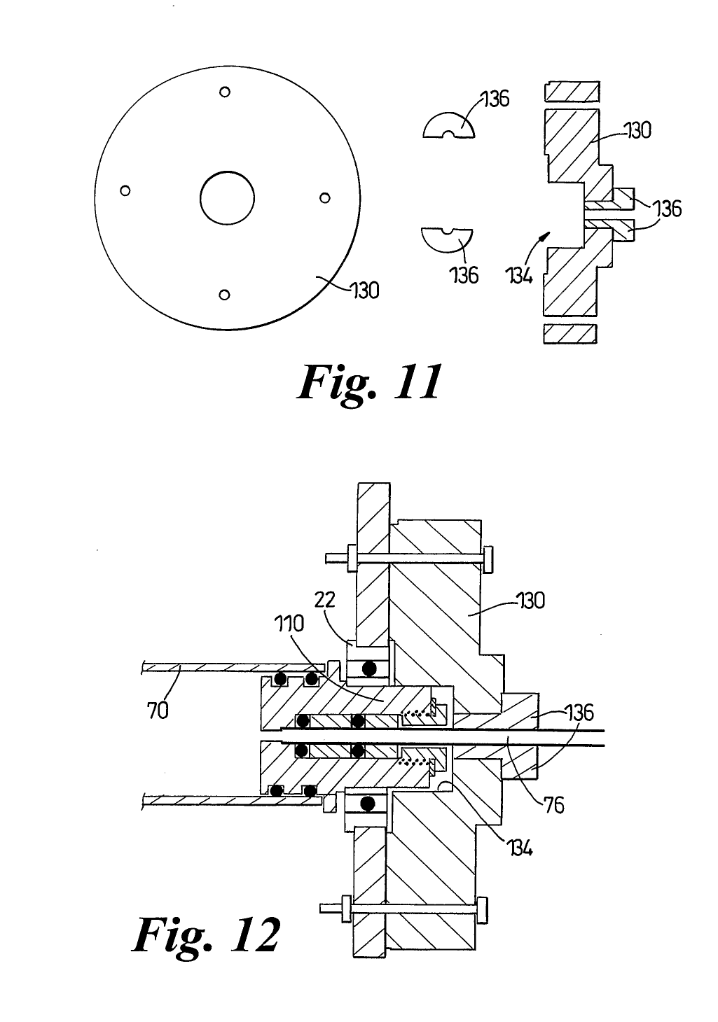

FIG. 11 is an exploded view of a needle clamp component;

FIG. 12 is a detailed view showing the needle clamp arrangement in position adjacent the end cap of the centrifugal tube assembly;

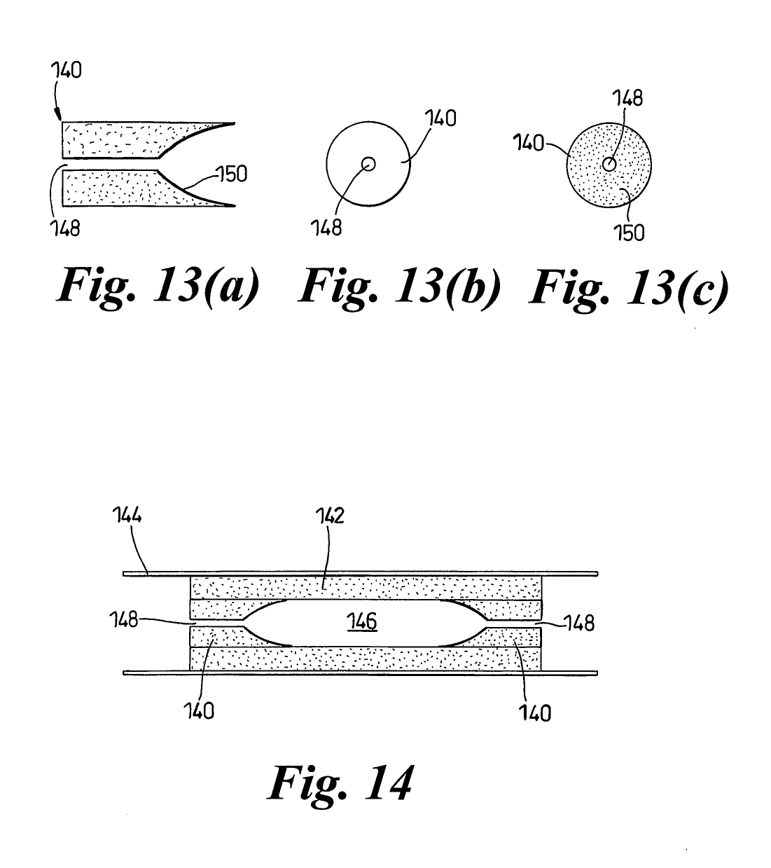

FIGS. 13 (a), (b) and (c) are side and opposite end views respectively of an insert for producing a tubular “mini-heart”;

FIG. 14 shows two of the inserts of FIG. 13 located within a centrifugal tube assembly;

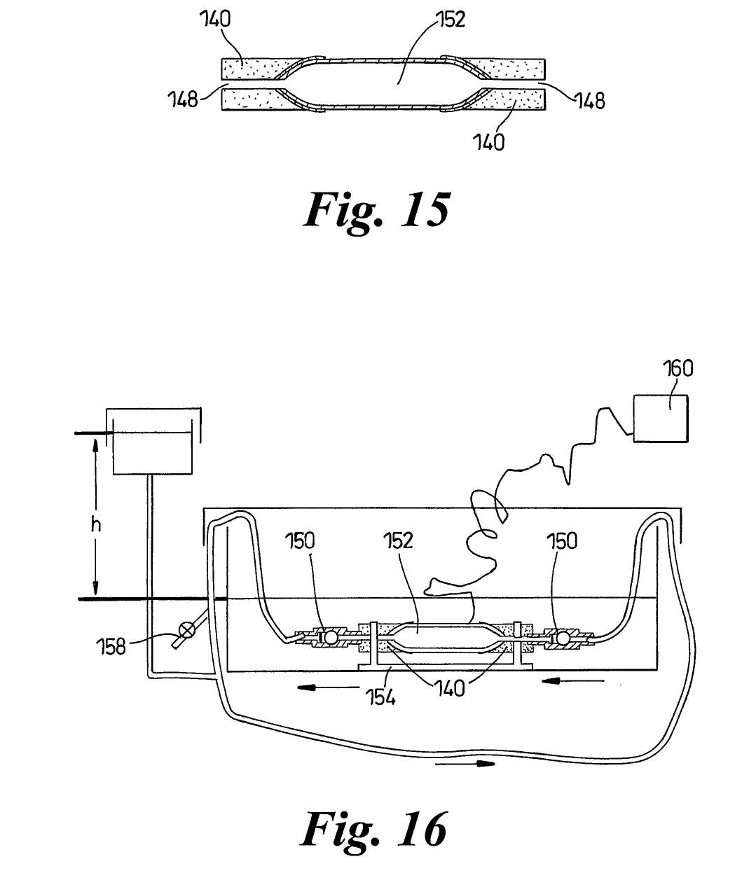

FIG. 15 shows the formed “mini-heart” and the inserts removed from the centrifugal tube assembly;

FIG. 16 shows the formed “mini-heart” set up in in vitro use;

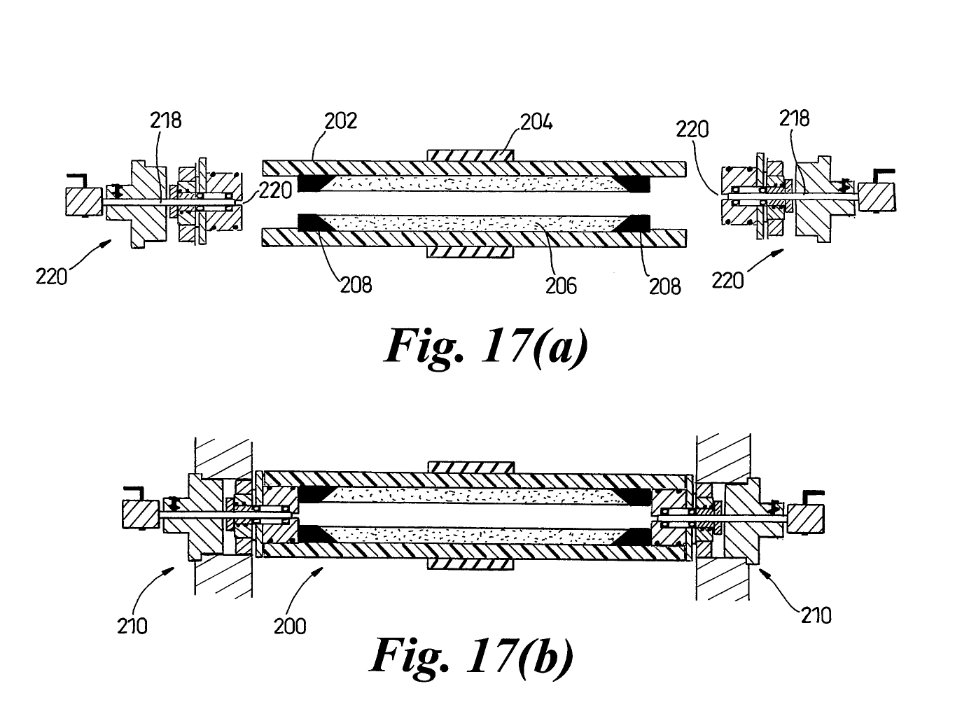

FIGS. 17 (a) and (b) are longitudinal sectional views through an embodiment of centrifugal tube assembly in accordance with this invention, with end caps removed and in place, respectively;

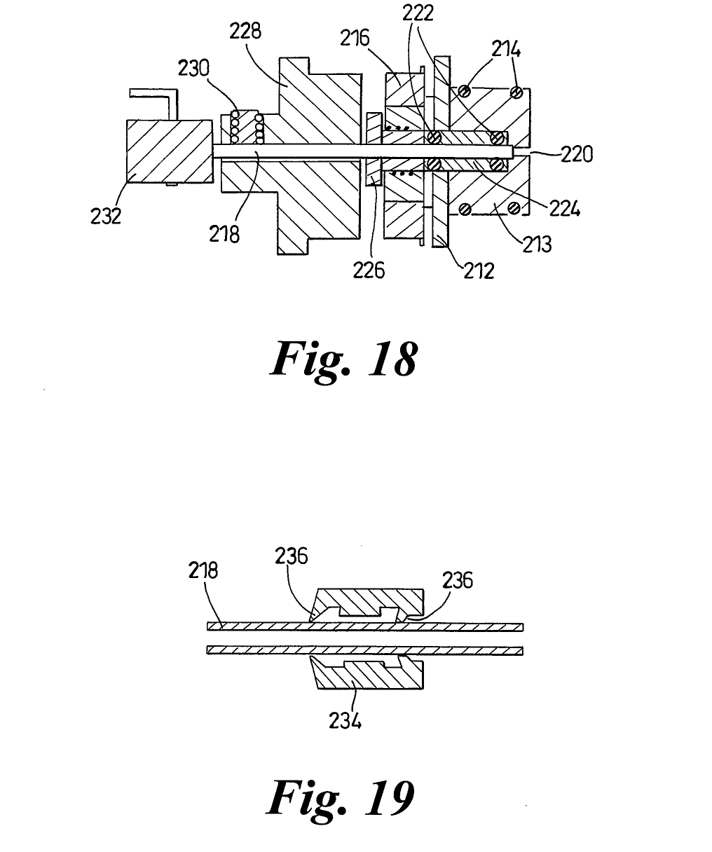

FIG. 18 is a section view through one end cap assembly of the arrangement of FIGS. 17 (a) and (b);

FIG. 19 shows an alternative seal arrangement intended to replace the ‘O’ ring seals and ‘O’ ring spacer of the arrangement of FIG. 18;

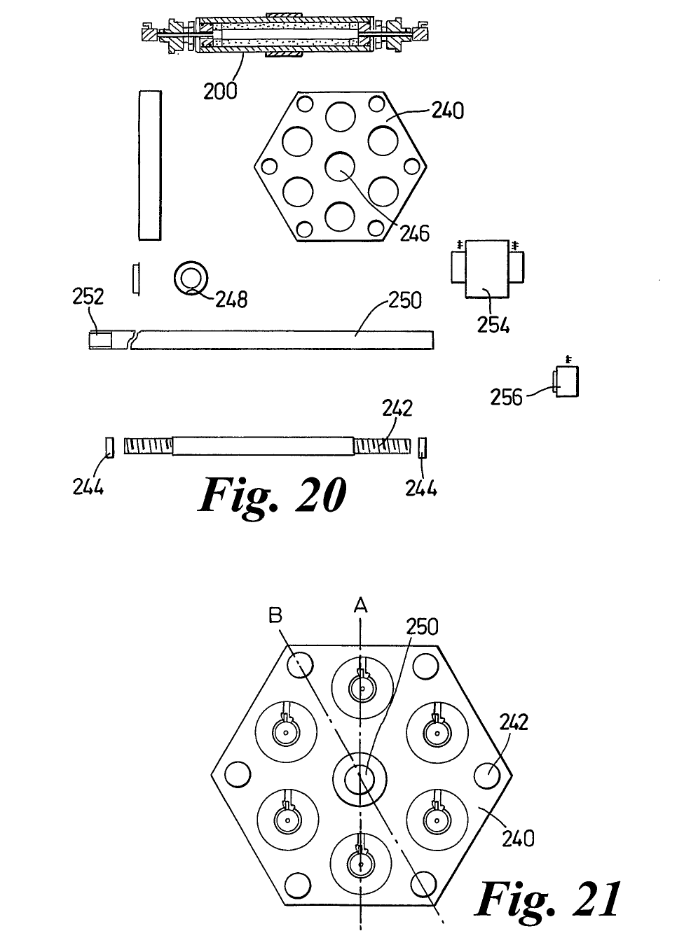

FIG. 20 is a schematic assembly view showing the various components that make up a hexagonal repeat unit;

FIG. 21 is a schematic end view of a hexagonal repeat unit;

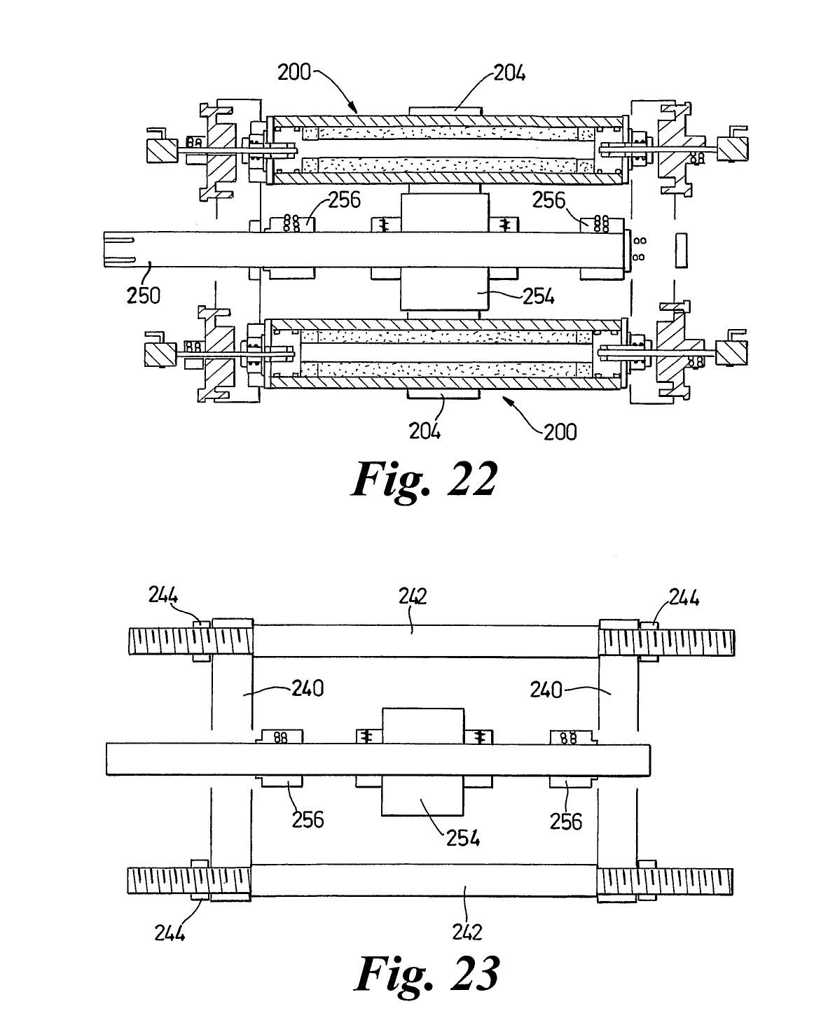

FIG. 22 is a section view taken on line A of FIG. 21;

FIG. 23 is a section view taken on line B of FIG. 21;

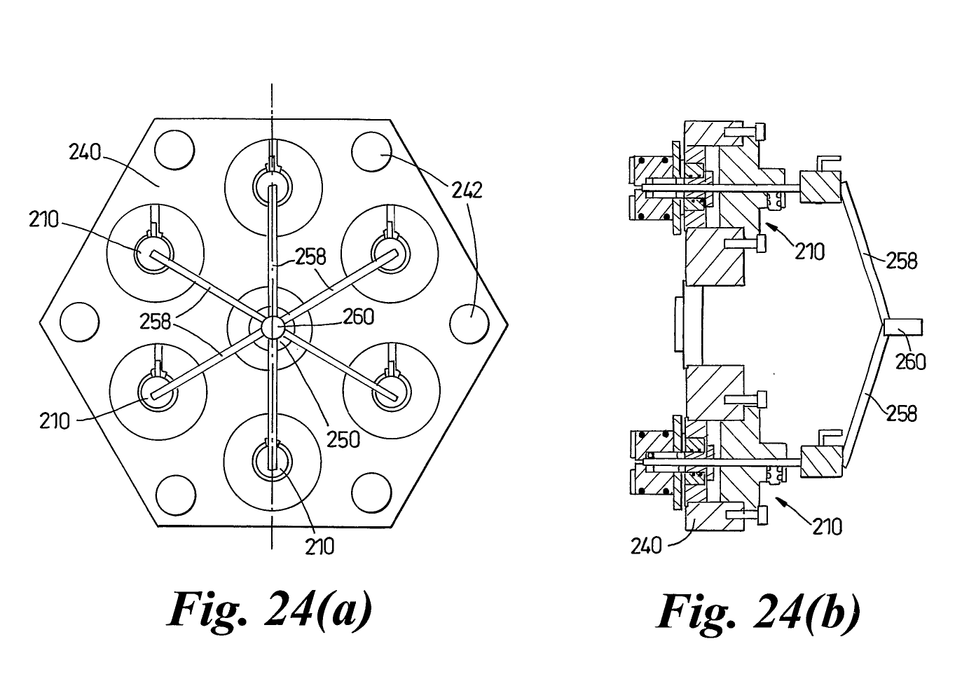

FIGS. 24 (a) and (b) are schematic end and side views showing the fluid connection tubes to the centrifugal tube assemblies making up the hexagonal repeat unit.

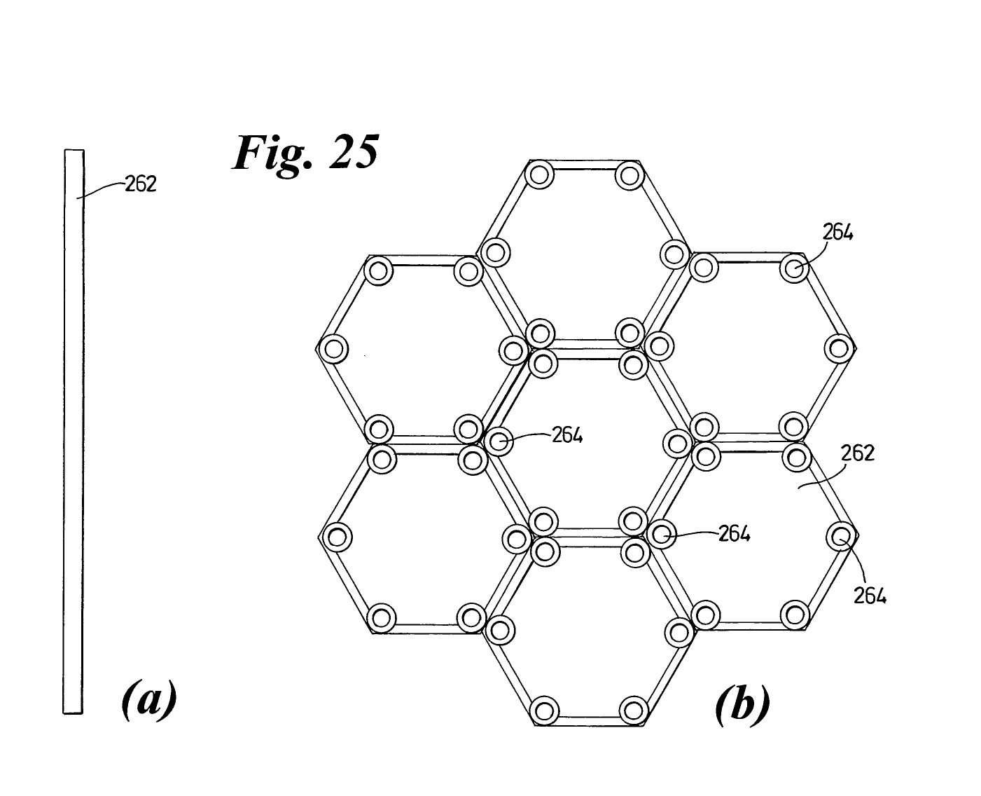

FIGS. 25 (a) and (b) are respective side and end views on a frame for holding seven hexagonal repeat units in an assembly to run forty two centrifugal assemblies at the same time;

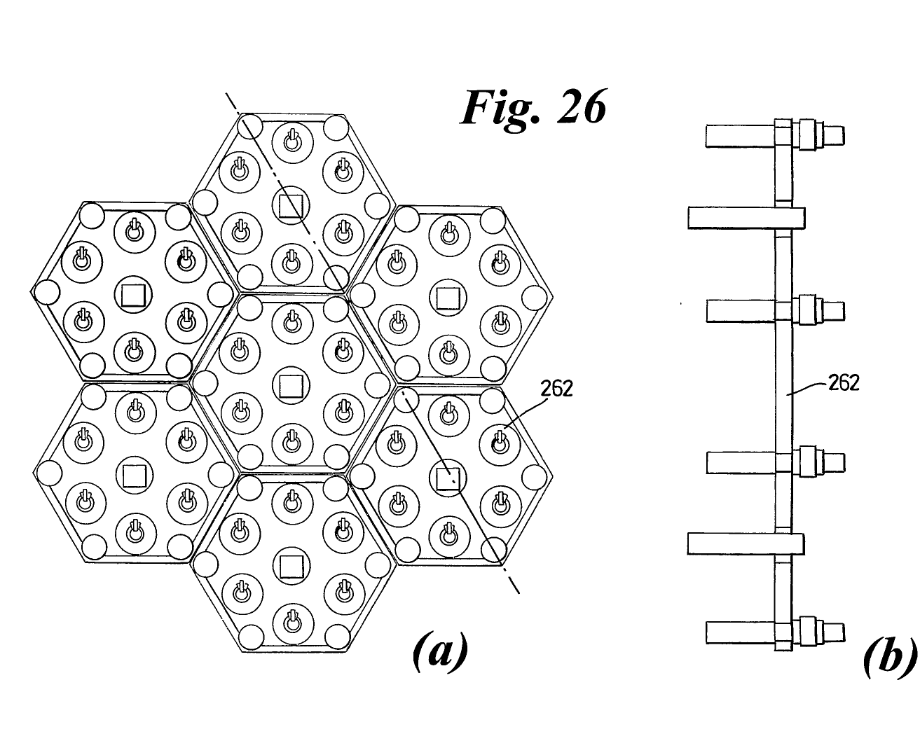

FIGS. 26 (a) and (b) are respective end and section views on an assembly of seven hexagonal repeat units held by the frame of FIGS. 25 (a) and (b);

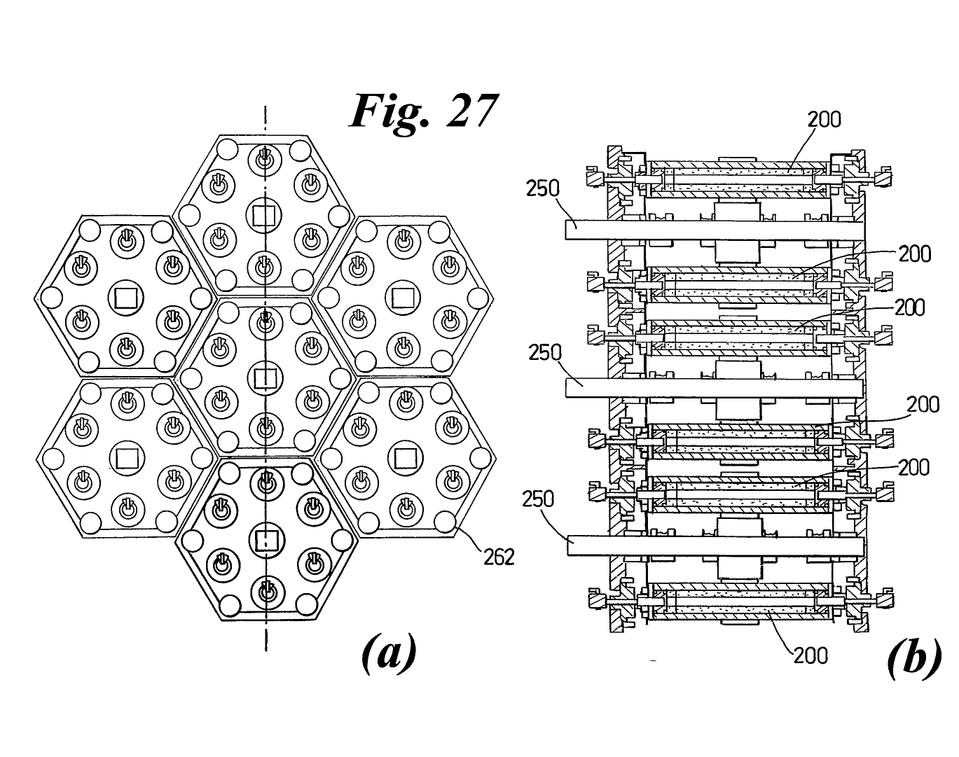

FIGS. 27 (a) and (b) are further end and section views on an arrangement with seven hexagonal repeat units;

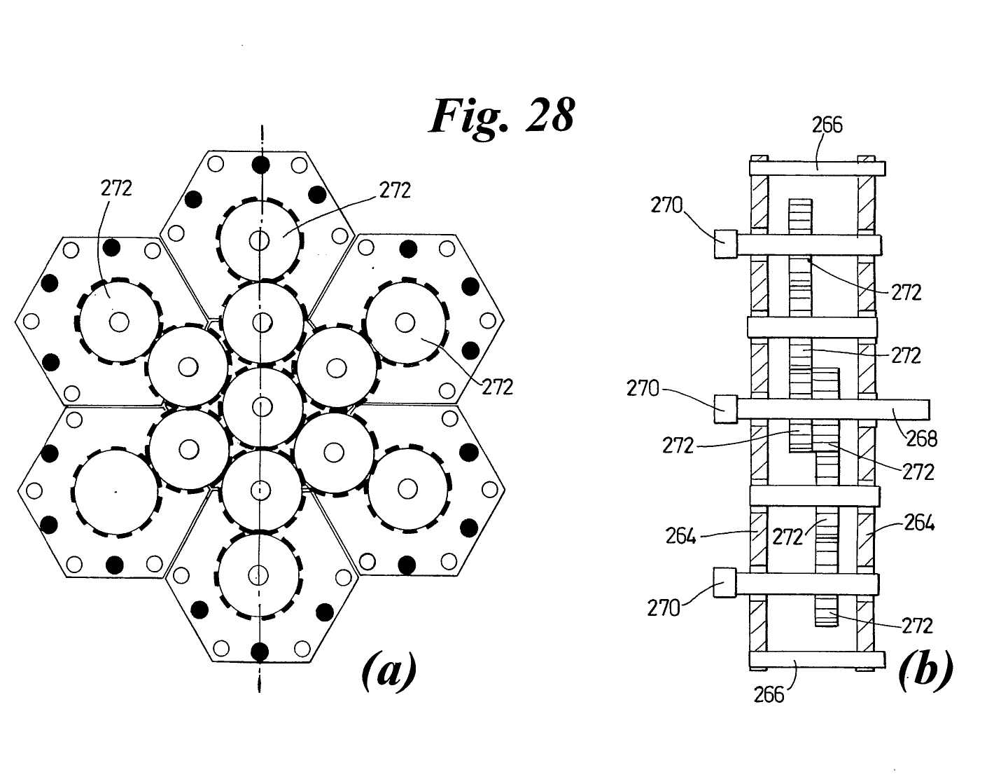

FIGS. 28 (a) and (b) are detailed end and section views through a multi-cog drive assembly for distributing rotational drive to each of the seven repeat units;

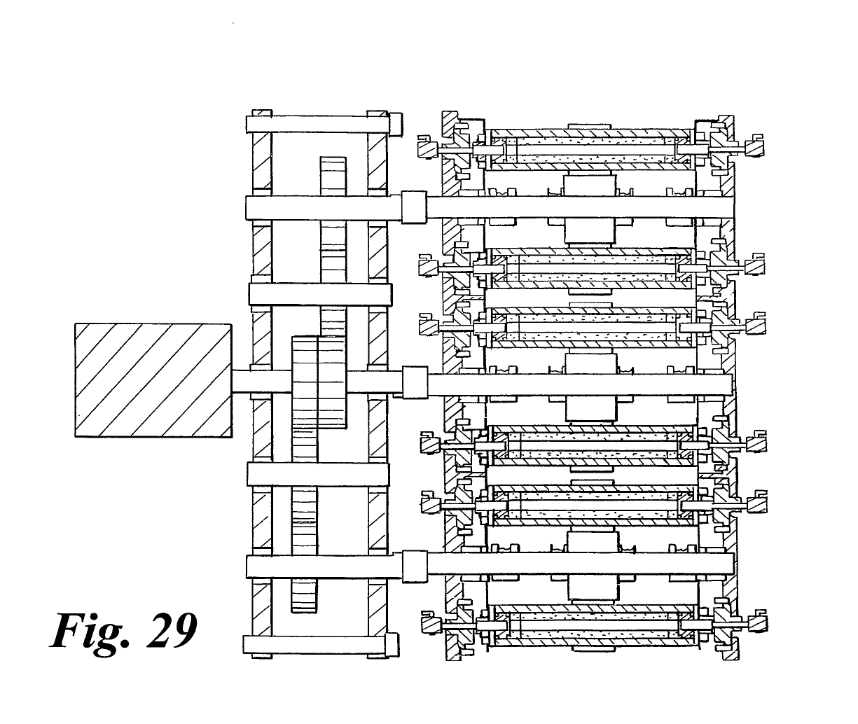

FIG. 29 is a side section view similar to that of FIG. 27 (b) but showing the common drive of FIG. 28 and a common motor;

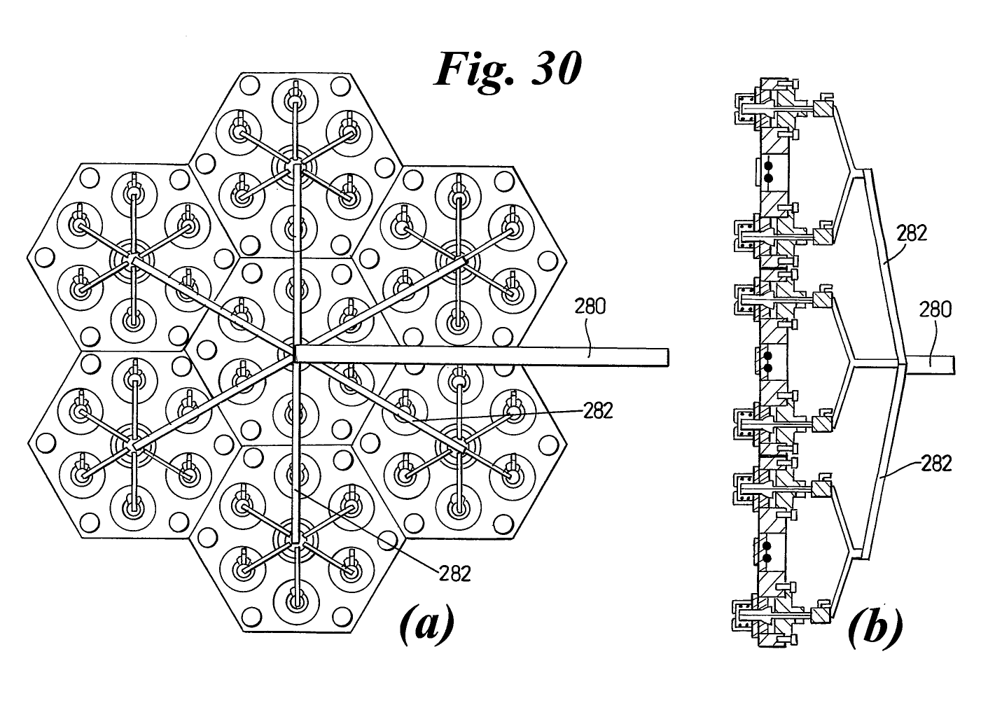

FIGS. 30 (a) and (b) are respective detailed end and side section views showing the fluid distribution network for directing fluid to and receiving fluid from each of the forty two centrifugal tube assemblies in the arrangement of FIGS. 25 to 29;

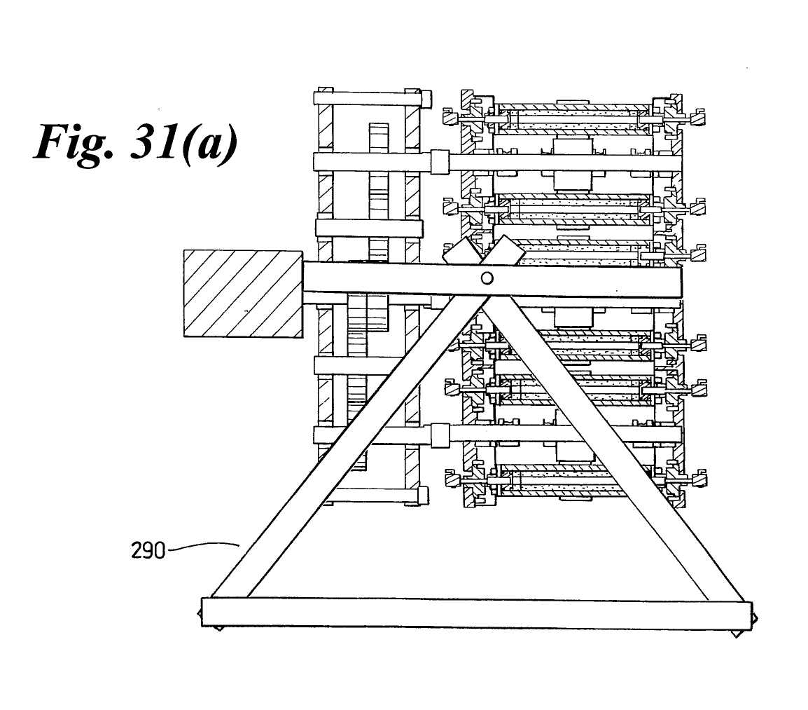

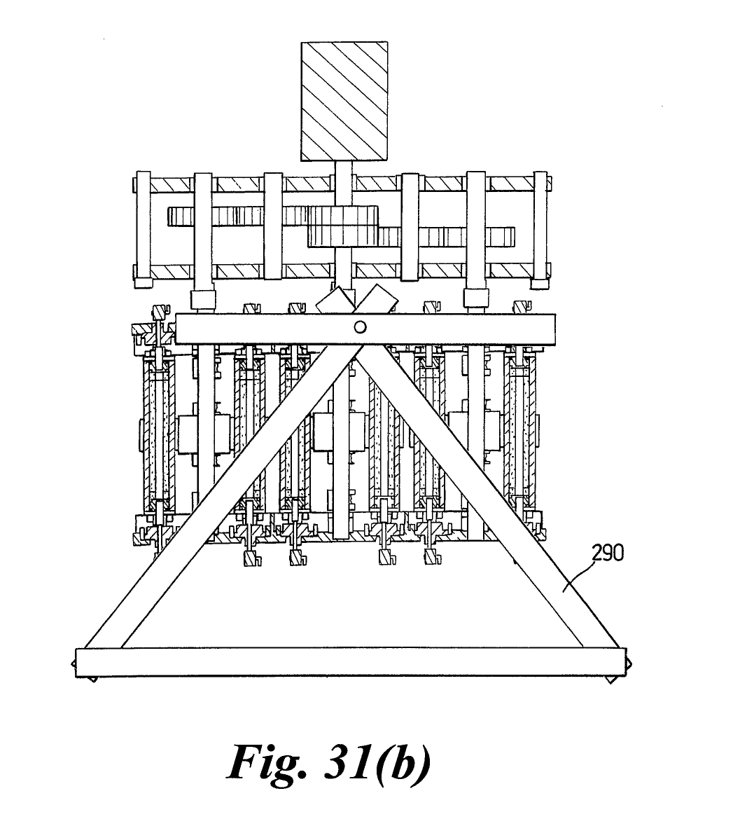

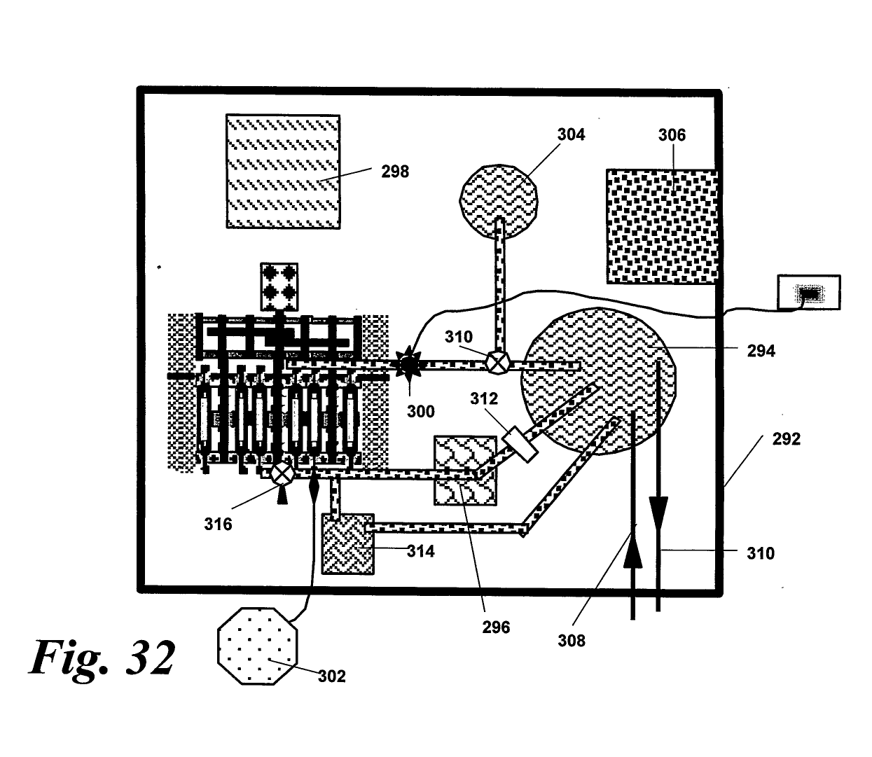

FIGS. 31 (a) and (b) are side views showing the assembly of seven hexagonal repeat units mounted in a frame so that they may be rotated in unison from the position of FIG. 31 (a) in which the centrifugal tube assemblies are horizontal, and the position of FIG. 31 (b) where the centrifugal tube assemblies are vertical;

FIG. 32 is a schematic top plan view of a cultivation assembly including the arrangement of FIGS. 25 to 30, with the tilting frame removed;

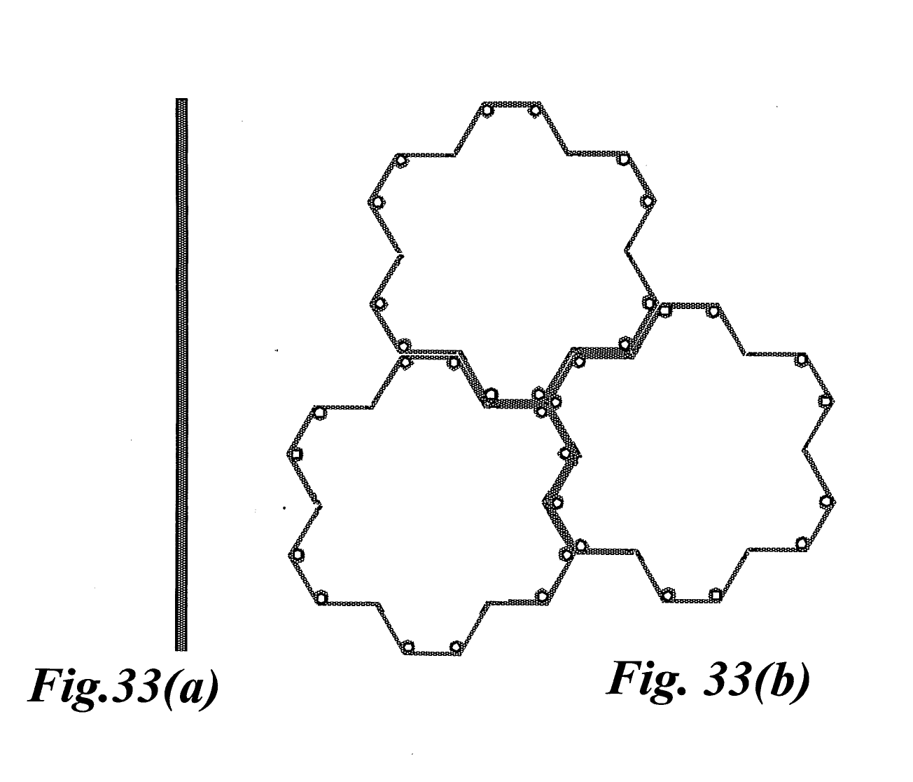

FIGS. 33 (a) and (b) are side and end views respectively on a frame construction showing how three of the assemblies of FIGS. 25 to 30 (themselves containing seven hexagonal repeat units) can be arranged to provide an array of one hundred and twenty six centrifugal tube assemblies;

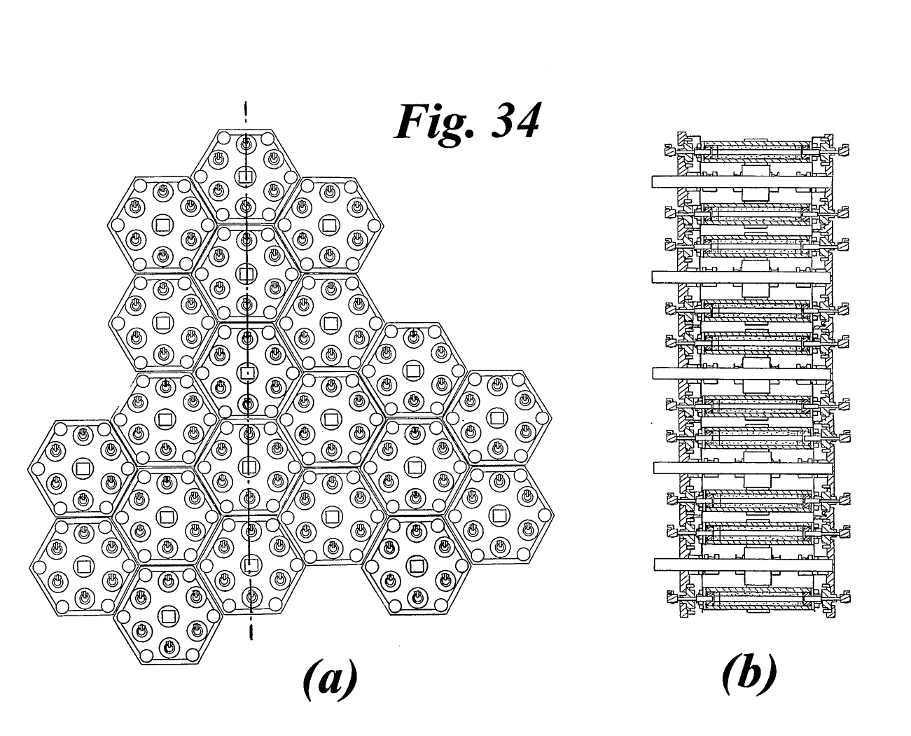

FIGS. 34 (a) and (b) are end and side section views respectively through an the arrangement made up of the frame of FIGS. 33 (a) and (b) with hexagonal repeat units installed;

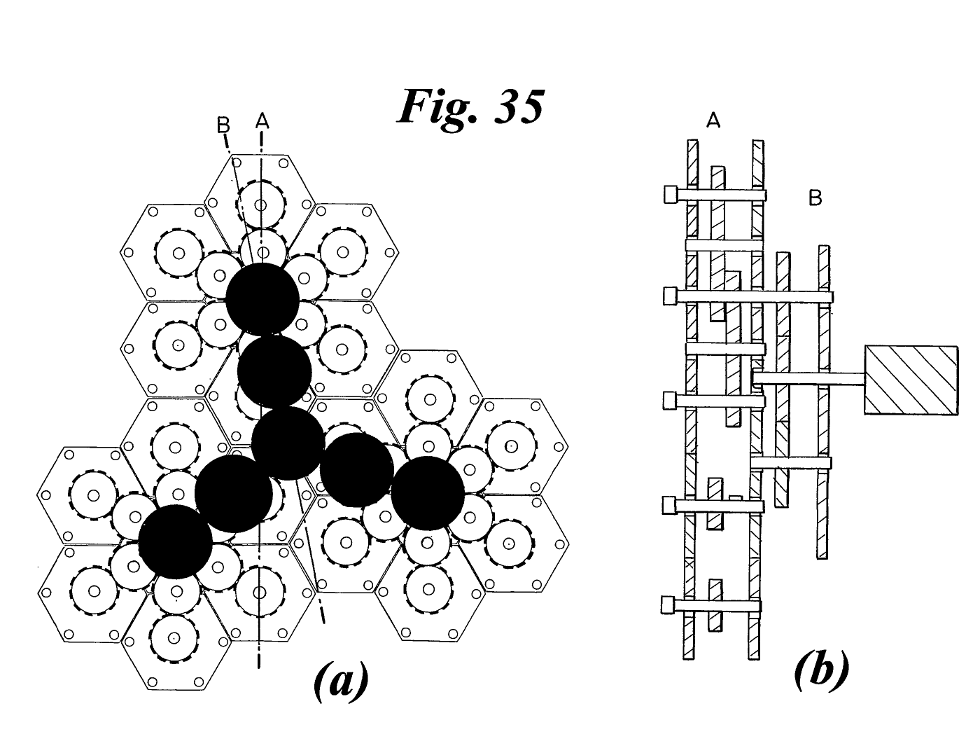

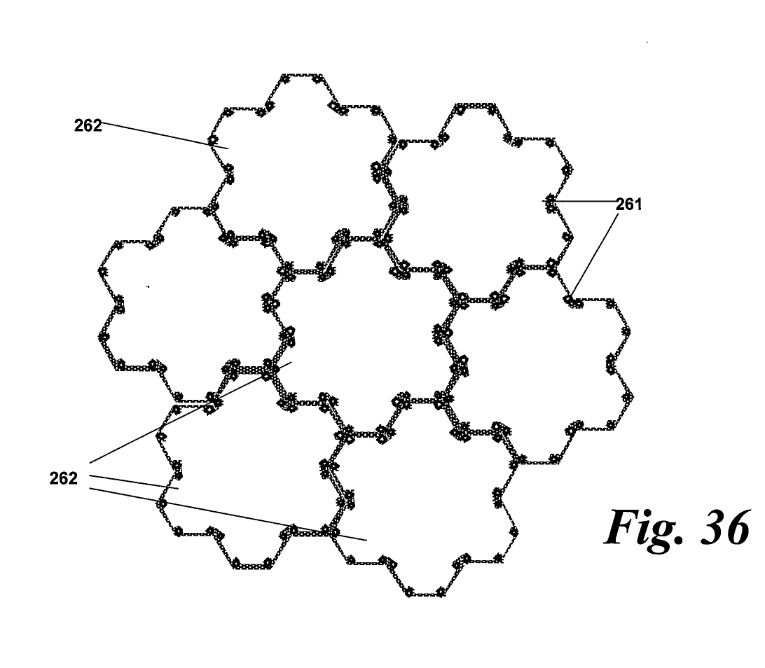

FIGS. 35 (a) and (b) are end and side section views respectively through a common drive arrangement for driving each of the centrifugal tube assemblies in this embodiment;

FIG. 36 is an end view of an end frame designed to hold seven of the assemblies of FIGS. 25 to 28 thereby providing an overall assembly of two hundred and ninety four centrifugal tube assemblies;

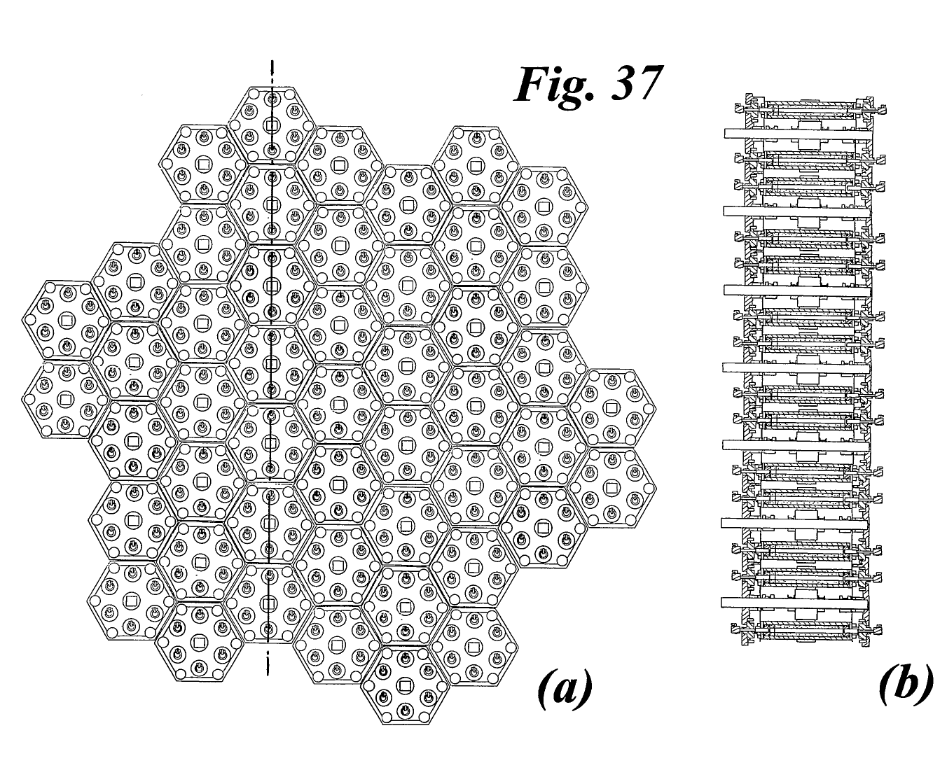

FIGS. 37 (a) and (b) are respective end and section views of the arrangement of FIG. 36 with the hexagonal repeat units installed.

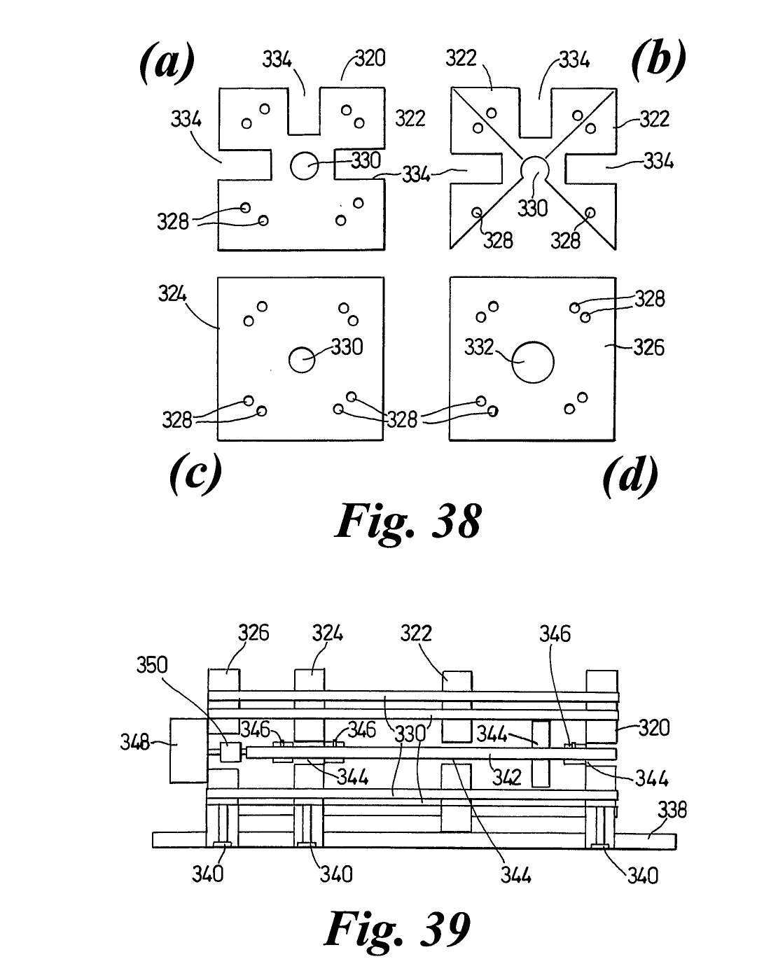

FIGS. 38 (a) to (d) are views of the four support plates for a further embodiment of apparatus for laboratory use for supporting three centrifugal tube assemblies;

FIG. 39 is a side view of the further embodiment of FIGS. 38 (a) to (d);

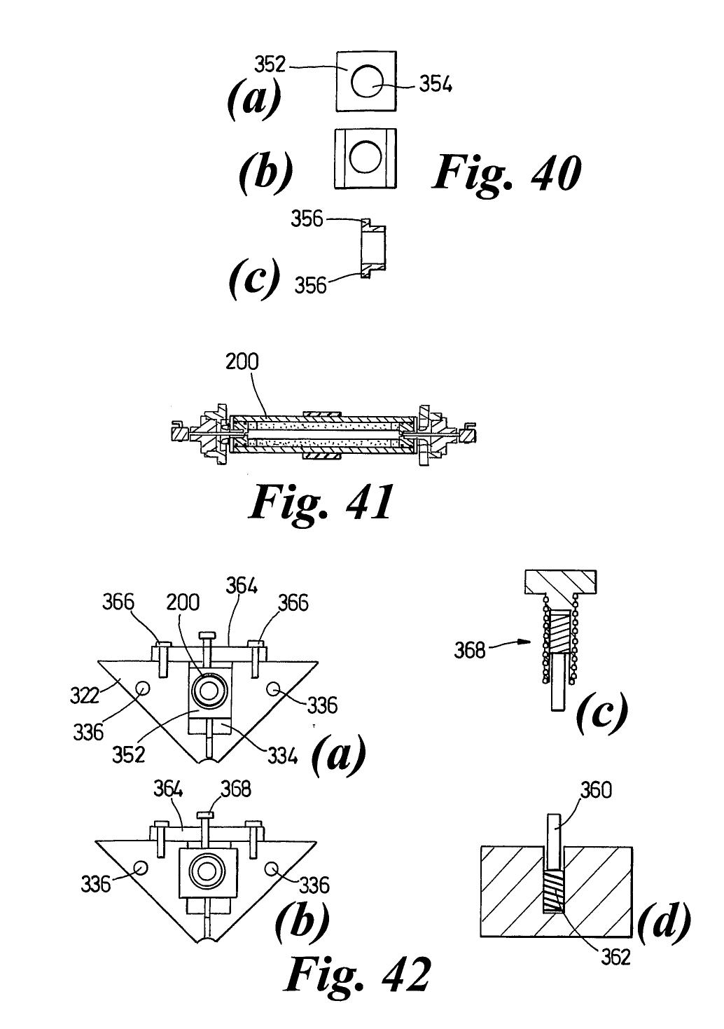

FIGS. 40 (a) to (c) show respective front and reverse side views and a top view respectively of a sliding holder for use in the further embodiment;

FIG. 41 is a view of a centrifugal tube assembly fitted between two sliding holders of FIGS. 40 (a) to (c);

FIGS. 42 (a) to (d) are respective views showing the mounting of the sliding holder on one of the support plates of the further embodiment;

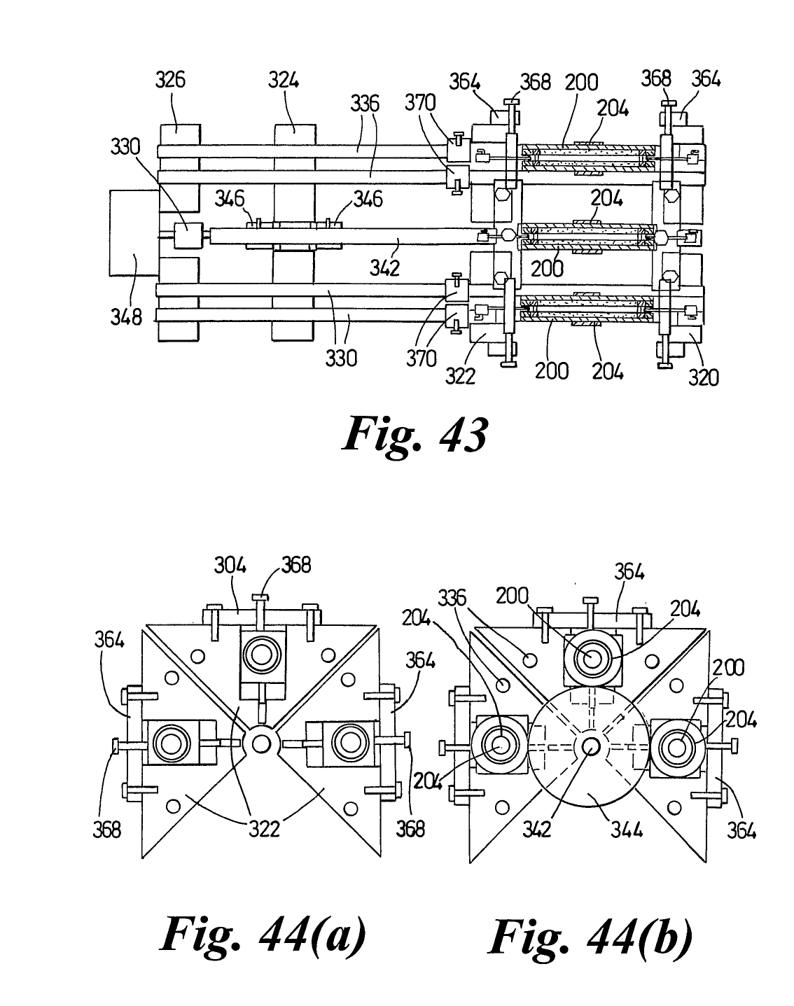

FIG. 43 is a top plan view of the further embodiment;

FIGS. 44 (a) and (b) are respective transverse views in the arrangement of FIG. 43;

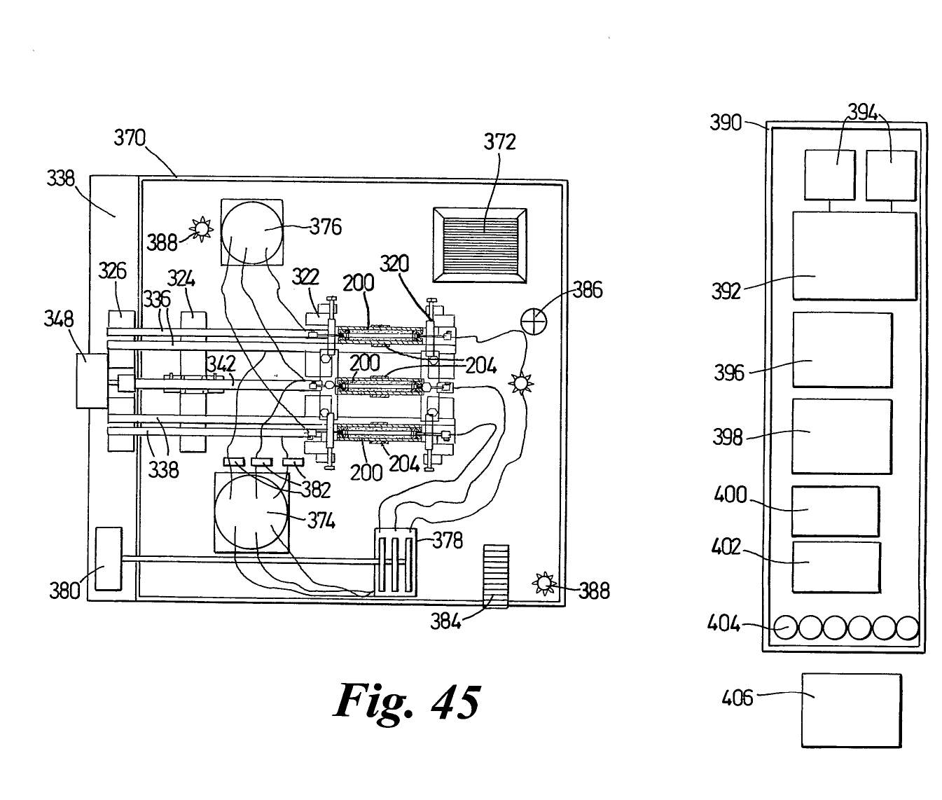

FIG. 45 is a schematic top plan view of the arrangement of FIGS. 38 to 44 and the associated control circuitry;

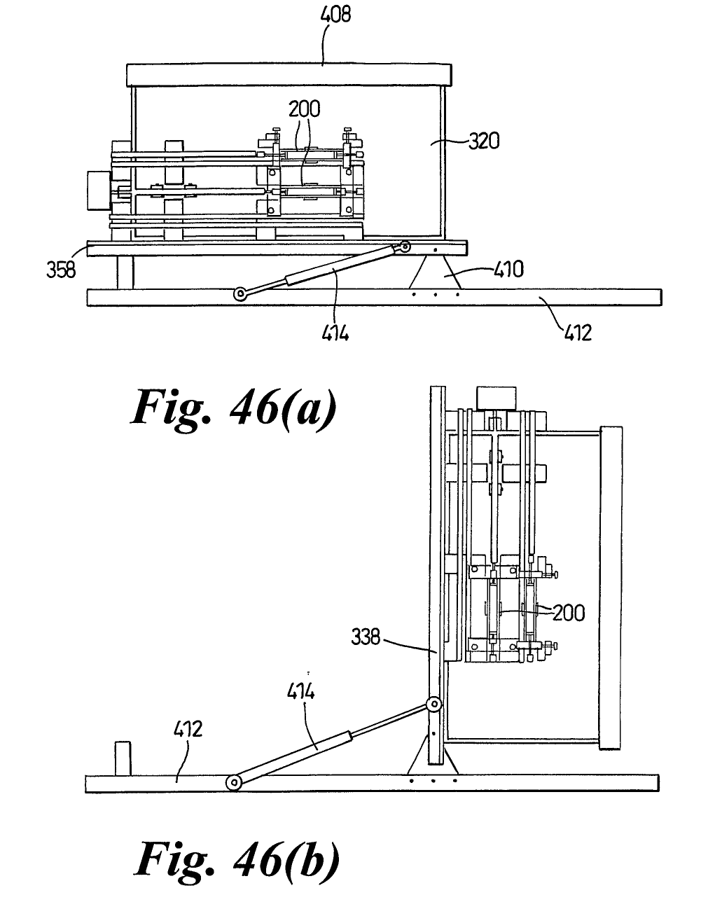

FIGS. 46 (a) and (b) are views of the arrangement of FIGS. 38 to 45 on a tilting cradle in the horizontal and vertical positions respectively;

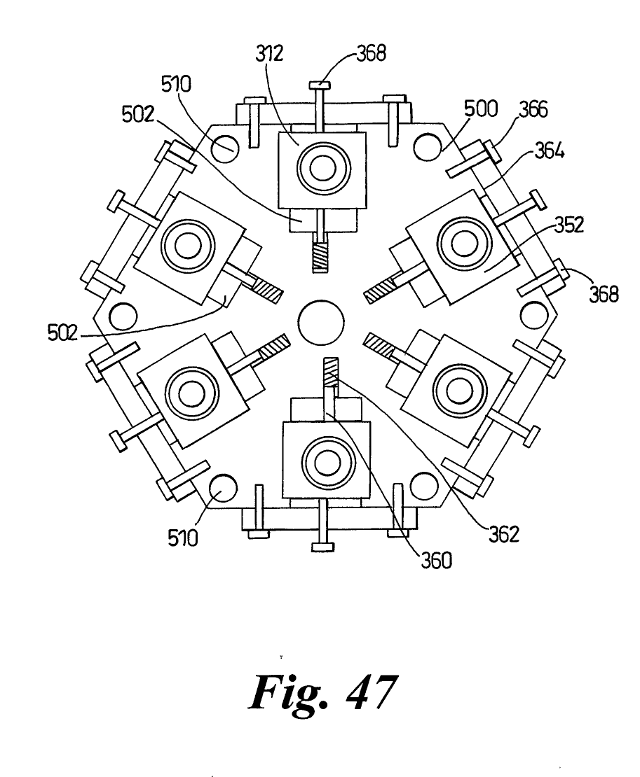

FIG. 47 is a section view on an arrangement of a further embodiment comprising a hexagonal array of removable centrifugal tube assemblies;

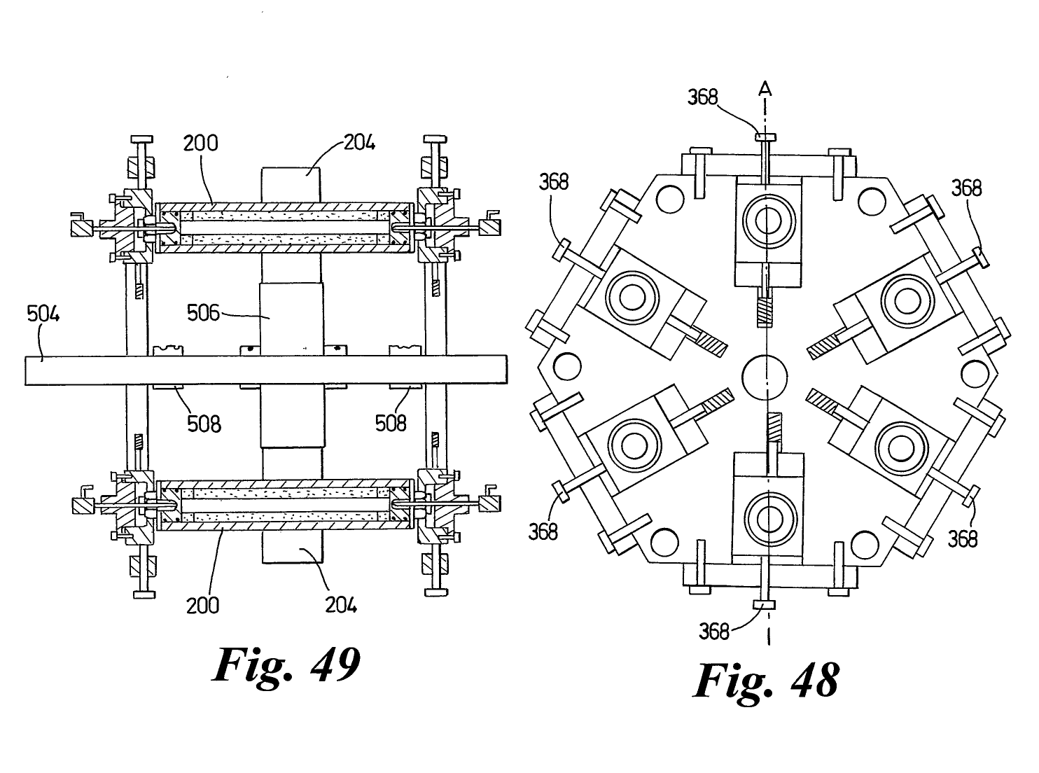

FIG. 48 is a displaced end view on the arrangement of FIG. 47;

FIG. 49 is a side view on the arrangement of FIG. 47;

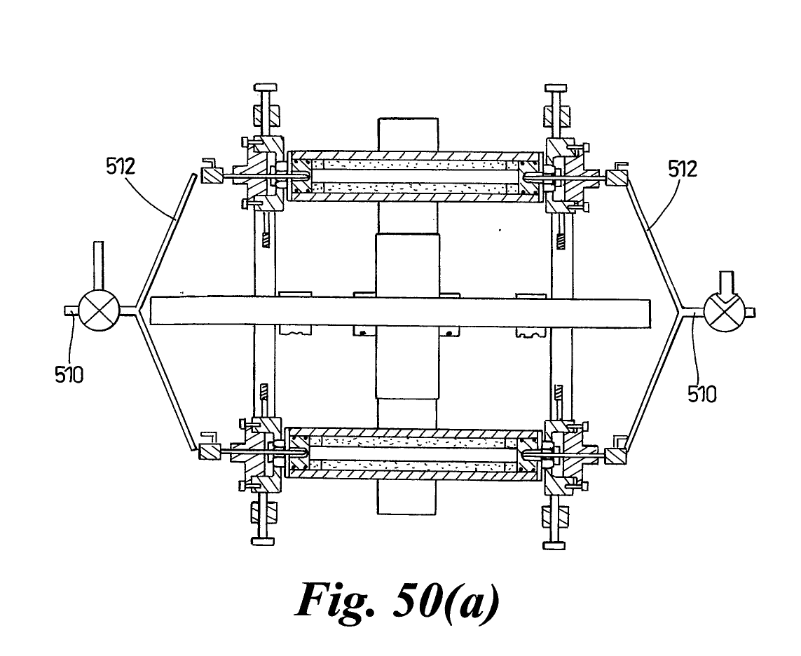

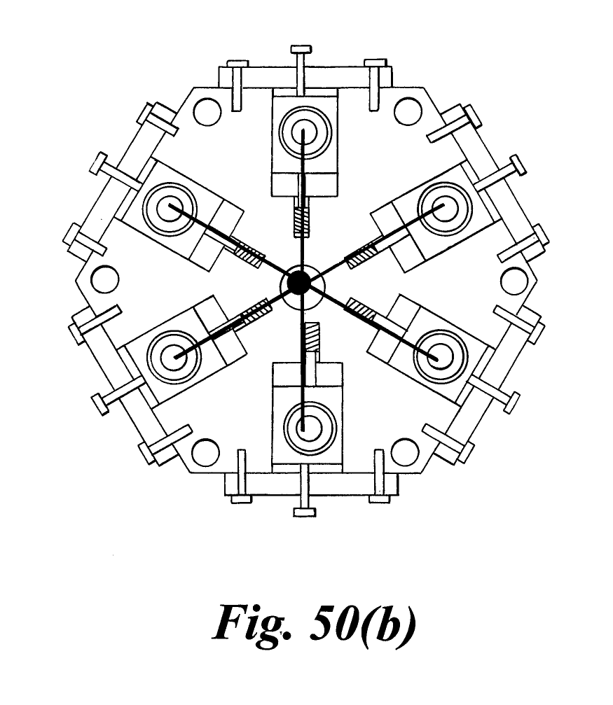

FIGS. 50 (a) and (b) are sides and end views respectively on the arrangement of FIG. 47 showing the fluid distribution;

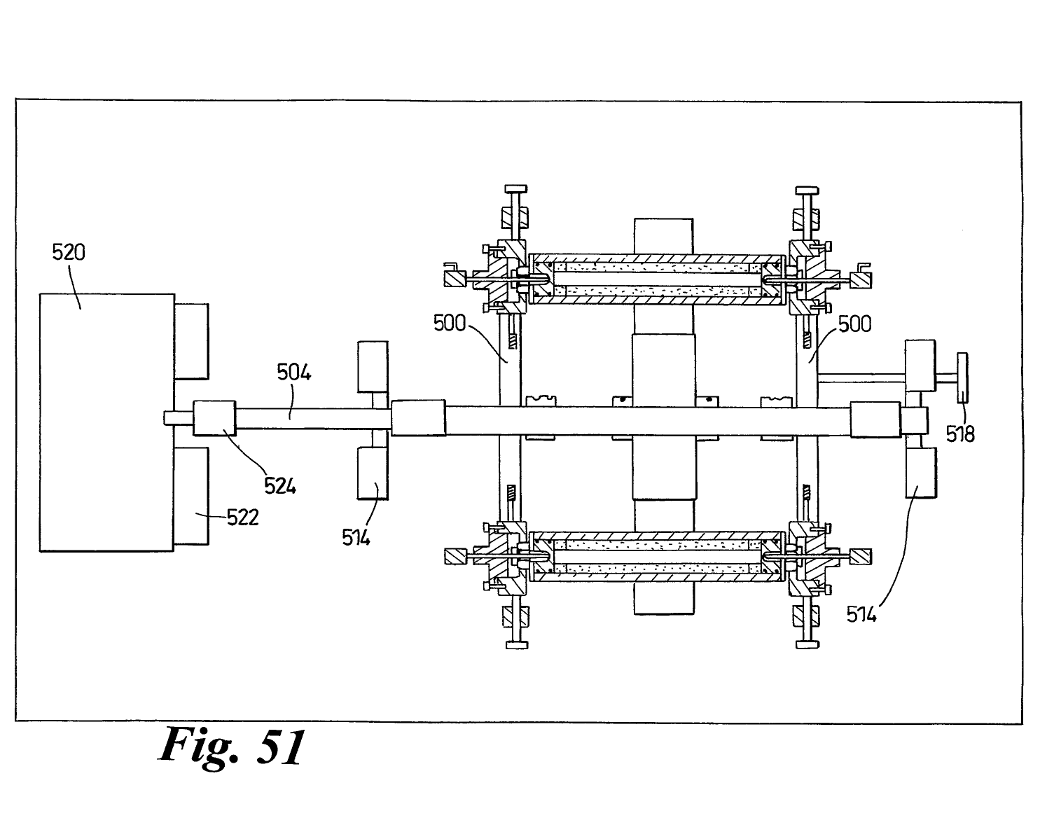

FIG. 51 is a top plan view of the arrangement of FIGS. 47 to 50 with a drive motor attached;

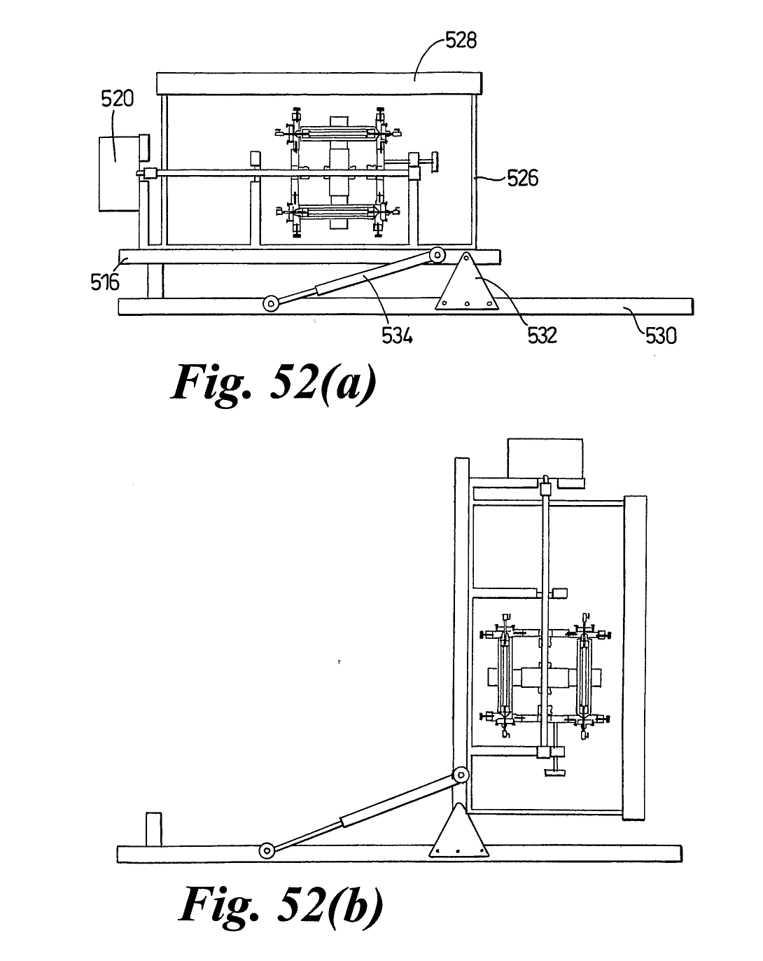

FIGS. 52 (a) and (b) are views of a tilting cradle arrangement showing the apparatus of FIGS. 47 to 51 in a horizontal and vertical position respectively, and

FIG. 53 is a schematic top plan view showing the apparatus and associated control circuitry.

Referring to FIG. 1, the apparatus of this embodiment comprises a base 10 on which is mounted a fixed bearing plate 12 and a facing, sliding bearing plate 14. The bearing plates 12 and 14 each house two bearings, namely respective first bearings 16, 16′ for rotatably mounting a lay shaft 18 connected to a variable speed electric motor 20, and respective bearings 22, 22′ for rotatably supporting a direct injection centrifugal tube assembly 24 for rotation. A pulley 26 is secured to the lay shaft 18, between the bearings 16 and 16′, and drives, by means of a drive belt 28, a drive pulley 30 on the centrifugal tube assembly 24. The centrifugal tube assembly 24 and its contents and components will be described in more detail below.

A fluid flow circuit is set up to cause culture medium to flow through the centrifugal tube assembly 24. The circuit comprises a reservoir 32 of tissue culture medium, having an inlet 34 for sterile 5% CO₂ in air for aerating the liquid tissue culture medium contained in the reservoir 32. The reservoir 32 also includes a vent with an air filter 36. The tissue culture medium is drawn from the reservoir 32 via an outlet tube 38 to a peristaltic pump 40 whence it is supplied by a discharge tube 42 to a 3-way tap 44 via a pressure-activated valve 46. The 3-way tap 44 is also connected to a cell injection syringe 46 and is operable to connect either the syringe 46 or the discharge tube 42 to an inlet coupling 48 to pass to the inside of the centrifugal tube assembly 24. Downstream of the centrifugal tube assembly 24, the flow path passes via an outlet coupling 50 to an outlet tube 52 defining an outlet path for the tissue culture medium which passes via a pressure measuring device 54 and a pressure-activated valve 56 to a 3-way tap 58. The 3-way tap may be used to direct the flow either to a waste vessel 60 which is provided with a vent and an air filter 62, or back via return tube 64 to the reservoir 32. Between the outlet coupling 50 and the pressure measuring device 54, there is a T-junction in the tube 52 where it is connected to a pressure pulse generator arrangement 66 which is described in more detail below.

The apparatus illustrated in FIG. 1 therefore provides an arrangement which supports a vessel for rotation at variable speeds defined by the variable speed motor 20 and through which a tissue culture medium may be caused to flow in controlled manner, and into which an initial cell suspension may be injected.

Referring now to FIG. 2, the centrifugal tube assembly 24 will now be described in more detail. The assembly comprises a hollow tube 70 made from, e.g. clear or opaque plastic, stainless steel or any other suitable material, and is fitted with the drive pulley 30 around the middle of its outer wall. Special end caps 72 are fitted to the opposite ends of the tube and can be made from, e.g. plastic, PTFE, or any other suitable material. The end caps have a bore 74 through their middle, through which passes a stainless steel blunt syringe needle 76 of a suitable gauge (typically between 14 and 19 gauge). The gap between the needle 76 and the hole 74 in each end cap 72 is sealed with two spaced ‘O’ rings 78. An airtight seal between each cap 72 and the respective end of the tube 70 is achieved with an ‘O’ ring seal 80. The end caps are constructed so that they are a tight fit within the tube bearings 22, 22′. The end caps 72 are held in place on each end of the tube by friction, or by any other suitable mechanism. The syringe needles 76 terminate outwardly in the couplings 48 and 50 respectively. The needles 76 are held fast against rotation by small clamps attached to the bearing housings (illustrated in FIG. 12 and described below).

The dimensions of the tube 70 can vary according to the size of the tissue-engineered vessel being constructed. For many purposes the tubes will have a typical internal diameter of between 5 and 25 mm, and a typical length of from 25 to 200 mm, but any size of tube deemed suitable can be used.

Referring to FIGS. 3 and 4, the tube is fitted with a plastic foam insert 54. The foam insert can be made from any non-toxic foam material. The insert is dimensioned to fit exactly within the tube between the end caps, as seen in FIG. 4, allowing no movement of the insert within the tube. The inner bore of the foam insert can have any diameter that is at least 2 mm less than the internal diameter of the tube 70.

The purpose of the foam insert 54 is to allow an increase in the internal diameter of the central bore when internal pressure is applied. When the internal chamber defined by the bore of the insert is pressurised, the foam will be compressed, increasing the diameter, and when the pressure is released the foam will return to its original uncompressed shape, thereby restoring the original diameter. This cycle of compression and decompression will be repeated many times and the foam is selected to be resilient enough to endure this and to return to its original shape after each compression. The invention is not limited to use of foam and any other suitable material or mechanism can be used which gives a similar effect.

Referring now to FIG. 5, if required, the inside of the foam insert 54 can also be lined with a thin layer of agarose gel. FIGS. 5 (a) to (e) show a way in which the layer of agarose gel may be formed, using a plastics core former 82 (FIG. 5 (a)). To do this, the tube 70 is assembled with one end cap 72 fitted and the foam insert 54 in place (FIG. 5 (b)). The plastic former 82 is placed inside the tube as shown in FIG. 4 (c). The gap between the former and the foam is carefully filled with a suitable agarose mixture (FIG. 5 (d)) and allowed to set to form an agarose layer 84. After setting, the former 82 is carefully removed leaving the agarose liner layer 84 behind. The second end cap 72 is now fitted to seal the tube (FIG. 5 (e)).

Alternatively, a material other than agarose could be used, for example latex rubber.

If further structural support is required for the vessel, then scaffolding inserts may be added to the inside of the foam insert adjacent the inner surface of the insert 54 or the inner surface of the agarose layer 84 if this is provided. The scaffolds can be prepared from any suitable material including Dacron, collagen, PGA, PLA, PET, or any other suitable material. Generally speaking, the scaffolding should be in the form of a tubular structure that is the same length and a very slightly smaller outside diameter than the inside diameter of the insert 54 or agarose liner 84, as the case may be, to allow easy insertion of the tubular scaffold into the assembly.

Referring to FIGS. 6 (a) to (c) there is shown a typical structure of a scaffolding insert comprising a PET, PGA or PLA biofibre spiral 86 with 6 longitudinal bracing fibres 88. Biofibre spirals 88 can be prepared with or without lateral support. Any type of suitable biofibre can be used (either biodegradable or non-biodegradable). For a vessel of 5 mm internal diameter, biofibres with a diameter of 20 to 50 microns would be suitable. The spacing between the coils may be of any suitable pitch, but would normally be between 200 and 1000 microns. In order to prepare the scaffold insert, a biofibre is wound around a former of the required diameter. If required, lateral biofibres 88 can be added, by weaving at least 6 biofibres between the spiral coils. The lateral support biofibres are woven inside and outside of each spiral loop as shown in adjacent coils in the views of FIGS. 6 (b) and (c). The lateral support fibres 88 should prevent possible cell contraction forces pulling the coils together and shortening the length of the vessel.

Referring to FIG. 7, the dimension of the biofibre scaffold insert 86 should be of the same or similar length and diameter as the central tubular hole within the foam insert 54 or the agarose layer 84, as the case may be. The scaffold structure is slid into position before fitting the second end cap.

Referring now to FIG. 8, at some point in the process the inside of the centrifugal tube 70 is subjected to pressure pulses. The size and rate of these pulses should eventually correspond roughly to those expected in the environment to which the tissue engineered material would be subject. Thus for a human arterial blood vessel, the size and rate of the pulses should eventually correspond roughly to those in the human artery. Accordingly, a pressure pulse generator is provided to provide a pulsed environment within the tube.

In FIG. 8, a pressure pulse generator is shown which consists of a variable speed motor 90 (FIG. 1—not shown in FIG. 8). The variable speed motor 90 drives a shaft 92 with a cam profile 94 fitted to the shaft. The cam profile 94 rotates and activates the plunger 96 of a syringe 98 via a sliding cam follower 100. The cam follower 100 is urged into engagement with the cam profile 94 by means of springs 102 on the supports on which the sliding cam follower slides, and a spring 104 urging the plunger 96 outwardly of the syringe 98. When the plunger 96 is pushed into the syringe, medium is forced out of the syringe into the tube 106 joined by a T-connector to the outlet tube 52 from the centrifugal tube 24. Pressure-activated valves 56 prevent to either side of the centrifugal tube assembly 24 the escape of medium through the connecting tubes, so that the medium inside the centrifugal tube 70 becomes pressurised. When the cam profile 94 releases pressure on the syringe, the plunger 96 is forced back to its original position by the spring 104. When this happens, medium is sucked back into the syringe 98 from the connecting tube 106 and the pressure is relaxed. The time interval between the pressure pulses is controlled by the rotation speed of the motor and hence the cam rotation speed. Thus, for example, a speed of 60 rpm would give 60 pressure pulses per minute. The actual pressure pulse generated is determined by the cam profile—which determines how far and how fast the plunger 96 is pushed into the syringe 98—and the internal cross-sectional area of the syringe 98. The cross-sectional area of the syringe and the distance the syringe plunger is moved can be adapted to the internal volume of different sized centrifugal tubes 24, so that the desired pressure pulse is obtained. A pressure sensor 56 in the system can be used to measure the pressure pulses during the cycle.

Referring to FIG. 9, this shows a schematic view of a centrifugal tube 70 formed by machining from a solid rod to provide a cylinder with an integral drive pulley 30 thereon.

FIG. 10 shows a detailed view of an arrangement of an end cap 72 in which a main body portion is formed by machining from a solid rod of PTFE. Here the main body 110 is formed with two grooves 112 which receive respective ‘O’ rings 114 to seal against the cylindrical tube 70. The body 110 has a central stepped bore terminating in an orifice 116, the bore is stepped so as to provide an abutment 118 for the end of the blunt syringe needle 76 which remains stationary as the tube rotates. Two ‘O’ rings 120 are provided in the bore in the main body and spaced by respective sleeves 122, 124, with the sleeves being held in place, compressing the ‘O’ ring seals 120 to make a watertight seal, by a threaded locking collar 126 and a locking washer 128.

Referring to FIG. 11, there is shown, in exploded view, a needle clamp device designed to keep the needle stationary as the tube rotates. The needle clamp device 130 comprises a collar with a recess 134 for receiving the exposed cylindrical portion the main body 110 of the end cap. Split, centrally located needle holders 136 are located in the collar 132 to clamp about and hold the syringe needle 76 stationary in use, as the end cap rotates within recess 134.

The assembled needle clamp arrangement is shown in detail in FIG. 12. The collar 130 is bolted to the respective bearing plate 12,12′ to restrain it against rotation. The end portion 110 of each end cap is located in the respective bearing 22,22′ and protrudes into the recess 134. The needle 76, held stationary by the split needle holders 136, extends into the end cap.

The arrangement described allows the centrifugal tube assembly 24 to be rotated about its axis so as to produce G forces, typically of between 1 G and 20 G, whilst the needles 76 at opposite ends are held stationary. The pressure pulse generator allows the pulse pressurisation of the system to range from between 0 to 200 gms/cm² with a pulse rate of between 10 to 120/min.

In use, the entire assembly and the supply medium may be placed within a small customised incubator so that the temperature is maintained at 37° C. The medium supplies continually aerated with CO₂ in air. All of the components of the machine that need to be sterile may be sterilised by autoclave or by gamma radiation or any other suitable means.

### EXAMPLES

The apparatus should be connected together aseptically and primed with a medium supplied by the peristaltic pump 40. Cells at a high concentration are then injected via the 3-way tap 44 until they form a homogenous suspension within the chamber within the tube 70. The speed is then increased to the required speed (depending on the internal diameter of the cell chamber) so that a G force of 10 G to 20 G is obtained. The net effect is that the cells are forced radially outwardly to the boundary between the medium/tube insert where they form a uniform layer. The centrifugal tube is rotated at this high speed for about 30 minutes, before pumping medium through the system at about 10 ml/hour, whilst the tube is still rotating at a relatively high speed, (typically 5 G to 20 G), so that the cells are not disturbed by the flow of medium. When all the cells have joined up to form a tubular vessel the speed can be reduced.

After about 8 to 24 hours rotation, the second cell suspension is added to the tube in the same way as before and put through the same process. After a further incubation time of about 8 to 24 hours, the third cell suspension is added in similar fashion and the procedure repeated.

The formation of a blood vessel or any other tubular structure is usually carried out in two stages. For a blood vessel, the first stage is the formation of a tube with three distinct cell layers, e.g. fibroblast cells: smooth muscle cells: endothelial cells, which should take between 24 and 72 hours. The second stage is the maturation of the tubular structure whilst under the influence of a pulsed pressure system. This part of the process may take from 1 to 4 weeks. For all stages of the vessel formation, it remains in the same apparatus, with no handling, thus lessening the risk of contamination. The net outcome is that, after about 24 to 72 hours, the centrifugal tube is lined with three separate cell layers. The number of cells added can directly control the thickness of each cell layer. For example, in a typical blood vessel, the fibroblast layer would be about 150 microns thick, the smooth muscle cell layer about 350 microns thick, and the endothelial cell layer ideally would be only one cell thick.

If necessary, biofibres in various forms could be used to strengthen the tissue engineered vessel.

Once the three layer vessel has been formed it undergoes a maturation process. In this process the tissue engineered vessel is subjected to pulse pressure over a period of time. This allows the cells in the vessel to be placed under stress and strain, similar to that of an in vivo blood vessel. Under these conditions the cells should produce a matrix similar to that found in normal blood vessels.

The whole process of vessel formation and maturation is carried out in a single apparatus without having to remove or handle the vessel until the process is complete. This is a considerable advantage over existing methods.

In use the complete sterile centrifugal tube assembly 24 is removed from its protective wrapping; protective caps are in position covering the needle luer fittings. One end cap is placed into the inside of the bearing of the fixed bearing holder. The drive belt 28 is now placed in position on the pulley wheel 30 of the tube assembly 24 and the pulley 26 of the driveshaft 18. The sliding bearing plate 14 is now moved into position so that the second end cap fits within its bearing 22′. Sliding bearing plate 14 is now fixed in position by locking thumbscrews (not shown) and a cross-brace is applied at the top, between the plates 22,22′ and secured with thumbscrews. The needle holders are fixed in position so that the inlet and outlet needles do not rotate with the tube assembly 24. The tube assembly is now firmly in position and is ready for use. If required, the drive belt tension can be adjusted by a simple moveable pulley mechanism acting on the middle of the belt (not shown).

As noted above, at some point in the process the inside of the tube 24 is subjected to pressure pulses. The size and rate of these pulses should eventually correspond roughly to those experienced in vivo. To minimise any possible damage to the newly formed vessel, the pulse rate and pressure will be initially low, and increased gradually over a period of a week or more. Throughout this maturation period, pressures of 5 gms/cm² to 200 gms/cm² and pulse rates of 10 to 100 pulses per minute will be used. These pressure pulses are generated by the pressure pulse generator previously described or other suitable pressure generators may be used.

Having located the centrifugal tube assembly in position, the peristaltic pump 40 is connected to the inlet needle 48 of the assembly via previously sterilised tubing 42 and the 3-way tap 44. The other end of the pump is connected to the tissue culture medium reservoir 32, the medium in the reservoir being aerated with a mixture of sterile 5% CO₂ in air.

The complete assembly is then placed inside a small customised 37° C. incubator together with the tissue culture medium reservoir and the connecting tubes. The motors 20, 90 for the rotation of the tube and the pressure generator should be outside the incubator to avoid excess heat production.

When the whole apparatus is assembled, the apparatus is primed with the medium using the peristaltic pump 40 at its fastest rate. All air bubbles are excluded from the system before any cells are added. This may be achieved by reorienting the system so that the centrifugal tube is placed with its longitudinal axis vertical, so that the medium enters the bottom of the tube and exits from the top. When the system has been primed, it is ready for the introduction of cells.

### Example 1

### Injection of Three Different Cell Types into the Assembly to Form a Tissue Engineered Blood Vessel

Preparation of Cell Suspensions

The process of cell addition and maturation will be described for an apparatus of the type described above, with an outside diameter of 6 mm and an internal length of 25 mm. This provides an internal volume of 705 μl, and so to fill the tube assembly 24 completely, about 725 μl of cell suspension is needed. The volume in the connecting tube between the centrifugal tube assembly and the cell injection site should be kept to a minimum to avoid cell wastage.

The outer cell layer of the completed vessel was of fibroblast origin, the middle layer of smooth muscle cells, and the inner layer of endothelial cells. In order to get this arrangement the fibroblast cells were injected first followed by smooth muscle cells and finally endothelial cells. The thickness of each layer formed was determined by the number of cells present in each cell suspension.

The procedure will be described for a fibroblast layer about 150μ thick, a smooth muscle cell layer about 300μ thick and an endothelial cell layer about 1 cell thick. In order to get these ratios packed cell volumes of about 70 μl, 140 μl and 5 μl respectively will be required.

Of course as cells are introduced into the tube assembly 24 they form layers, then the free volume in the centre of the assembly 24 will be reduced. After the formation of the fibroblast layer, the free volume will be about 635 μl. Thus only 650 μl of smooth muscle cell suspension can be added to the assembly 24 for the formation of this layer. After the formation of the fibroblast and smooth muscle layers the free volume inside the assembly 24 will only be about 495 μl. This means that only about 510 μl of endothelial cell suspension should be injected into the assembly 24.

Cells suspension can be prepared either directly from tissues or from monolayer cell cultures. A suspension of fibroblast cells, with a packed cell volume of about 70 μl, was prepared from monolayers. The cells were washed twice in DMEM medium and re-suspended in about 725 μl of special spheroid forming medium (SPD medium). Reference is directed to our published PCT Application WO 00/78927 for a description of production of this medium. The tube assembly 24 was swiveled through 90° so that the assembly 24 was in a vertical position with the inlet tube 3-way tap 44 at the bottom. A syringe 46 with the 725 μl of fibroblast cell suspension was inserted into its luer fitting on the 3-way tap 44, and the cells injected. Cells in the tube between the assembly 24 and the injection site were pushed through with a small volume of the same medium. The assembly 24 was replaced in its horizontal position, the motor connected and the incubator lid closed. The assembly 24 was rotated at about 10 rpm for 1 minute, to make sure the cells were evenly suspended. The speed of the assembly 24 was then increased to about 2000 rpm, which is equivalent to a G force of about 14 G at the tube insert/cell suspension boundary. Under the G force the cells are quickly pushed to this boundary and remain in this position. After 10 minutes, the peristaltic pump is switched on so that medium flows through the centre of the assembly tube 24 at about 10 ml/hour. Any cells that were left in the connecting tubes were collected in the waste vessel 60 by diverting the flow there for the first hour by means of the three way tap 58.

After one hour the three way tap was operated to direct the culture medium to the reservoir 32 for recirculation. Also after one hour the rotation speed of the assembly 24 was reduced to about 1000 rpm, which is equivalent to about 3.5 G.

After 8 to 24 hours rotation of the assembly 24, the second cell suspension was added. The peristaltic pump 40 was switched off and the drive motor 90 also switched off. The assembly 24 was again swiveled through 90° and the process of cell injection carried out as before, except that this time 650 μl of smooth muscle cell suspension (with a packed cell volume of 140 μl) was used. After the cell injection sequence the assembly 24 was left rotating for a further 8 to 24 hours at 2000 rpm.

The third cell suspension was then added in exactly the same way as the second cell suspension, except that 510 μl of endothelial cells with a packed cell volume of 5 μl was used. The assembly 24 was left rotating for a further 8 to 24 hours, giving a total time of just over 24 hours for the formation of the 3 distinct layers of cells.

The cell types and quantities can of course be varied to give different cell layer thickness as desired. Rotation speeds of the assembly 24 can also be adjusted to give different desired G force conditions.

A variation would be to inject one cell suspension containing all the cell types required. For example 720 μl of cell suspension, containing 70 μl, 140 μl and 5 μl of packed cell volume fibroblasts, smooth muscle cells and endothelial cells respectively, can be prepared. This can be injected in one go giving all the cells required for the vessel formation. The cells may organise themselves spontaneously into the desired layer formation, making the injection of the different cell layers separately, unnecessary.

Maturation of the Three Layered Tube by Pressure Pulses

Use of the Pulse Pressure Generator

After the formation of the three cell layered tube over a 25 hour period the maturation process was carried out using the pressure pulse generator.

Blood pressure measured in the brachial artery under normal conditions is usually around 120/80 mm of mercury. 120 mm of mercury corresponds to a pressure of about 160 gm/cm² and this was used as an approximate upper limit of pressure used in the maturation process. The aim during the maturation process is to gradually increase the pressure pulses applied to the 3-layered vessel in the assembly 24 from about 5 gm/cm² (after layer formation) up to 160 gm/cm² at the end of the maturation period. The frequency of the pressure pulses should also be increased over this period of time from about 10 pulses/min to 80 pulses/min. The ideal length of time for maturation is expected to be between 1 and 4 weeks.

At the start of the maturation process, 10 pulses/min should be used at a pressure of 5 gm/cm². Both the pulse rate and pressure will be increased on a daily basis over the maturation period.

Any combination of increase in pressure and/or rate can be used and any length of time for maturation can be used, although, it is clearly desirable to keep vessel maturation to the shortest time possible.

During the maturation process the speed of rotation of the assembly 24 can be reduced (if desired) to about 500 rpm (which will give about 1 G in a 5 mm internal diameter vessel). In some variations it may be possible to stop the rotation of the assembly 24 altogether during the maturation process, but a low rotation speed should nullify any possible gravity effects.

The tissue engineered vessel can be removed from the assembly at any time during the above process, but will usually be after vessel formation and any time during the maturation period.

All the motors are switched off and the tubes are disconnected from the ends of the assembly 24. Sterile male needle caps are placed into the needle luer fittings. The assembly 24 is removed to a laminar flow hood and wiped over with 70% isopropyl alcohol and placed in a suitable clamp in a vertical position. The bottom needle luer cap is removed followed by removal of the top cap, and the fluid medium that comes out is collected in a suitable container. The top, and bottom end caps 110 are removed and the foam 54 or foam/agarose insert 54/84 is pushed out into a suitable sterile container containing the required tissue culture medium. The formed vessel can now be gently pulled out of the inserts. If this is not possible then the foam insert 54 can be cut lengthwise and gently removed from the formed vessel.

The vessel can now be used or processed for contraction and relaxation experiments, burst pressure measurements, histology, immunohistochemistry, electron microscopy or for any other purpose that is deemed suitable.

Examples of Tissue Engineered Products

Blood Vessels

Blood vessels of most diameters and length can be prepared by this method, by using different sized assemblies 24 and foam inserts 54. Larger vessels can also be prepared with an outer reinforcement of Dacron® or any other suitable material. Smaller vessels can be supported by scaffolds made of collagen, PLA, PGA or any other type of suitable biofibre material.

Ureters

By using smooth muscle cells and urethral cells it is anticipated that tissue engineered ureters may be made by this method, although much lower pulse rates and pressures would be used.

Bladders

By modifying the shape of the assembly 24 and replacing the pulsed pressure system with a slow change rate/large volume change inflatable system a tissue engineered bladder or part thereof may be made.

Vascularised Mini Organs

It is anticipated that vascularised mini organs may also be made using the method and apparatus as described. To do this an assembly 24 having an insert 54 with an internal diameter of about 2 or 3 mm can be used. The assembly is set up as described above, and a small number of fibroblasts is injected to give a cell layer about 50μ thick. When this layer has been formed, the cells required in the mini organ are injected. An example of a cell type that could be used would be insulin producing β cells from the pancreas. Enough cells would need to be injected to give a layer about 300μ thick. The cells for the blood vessel are injected next in the same sequence as described previously. However the cell layers in the vessel should be thinner; for example the structure might comprise a fibroblast layer about 10 to 50μ thick, a smooth muscle layer about 20 to 100μ thick and a single cell layer of endothelial cells. After maturation for a few days to a week (under lower pressures than a 5 mm internal diameter blood vessel), the mini organ can be removed from the assembly 24. It should be in the form of a vessel with 5 layers, a thin outer layer of fibroblasts and matrix, a layer of β pancreatic cells and three inner layers representing the blood vessel cells. The total thickness of the mini organ wall would be about 350 to 500μ, which should give the inner blood vessel an internal diameter of 1 to 2 mm, depending on the insert size used.

These mini organs could be used for in-vitro studies or even in-vivo studies, if they were joined up to an animal's blood supply.

Sheets of Cardiac Muscle

In another arrangement a sheet of cardiac muscle may be produced by forming a tubular structure of cardiac muscle cells using the centrifugal tube assembly described above, but without the need for pulsed pressure. The formed tube of cells can then be cut along its length to give a rectangular sheet of cardiac muscle. This sheet of cardiac muscle could be useful in cardiac repair.

Preparation of a Mini-Heart

Referring now to FIGS. 13 to 16, in another arrangement, the centrifugal tube assembly is fitted with special shaped inserts 140 which fit within a foam/latex cylinder 142 located in the rotating tube 144 of the centrifugal tube assembly, as shown in FIG. 14. In this way is provided a growing space consisting of an enlarge chamber 146, communicating at each end with smaller diameter inlet/outlet tubes. The inserts 140 may be made of silicon rubber with the inner, concave surfaces defining the ends of the chambers 146 being covered by a structure 150 forming a fine micro-fibre cone bonded to the silicon rubber. The micro-fibre cone may be made of any suitable fine fibrous materials that cells will attach to (including PGA, PLA, Dacron etc.). The fibrous cone 150 may be pre-coated with cell adherence factors; such as fibronectin or serum etc.

In use, the inserts are placed inside each end of the foam/latex insert 142, as seen in FIG. 14 and the end caps (not shown) are pushed into the ends of the centrifugal tube 144 as previously described in the arrangements above, with the caps flush with the foam/latex insert. The centrifugal tube assembly is then set up in the apparatus described above. A cardiac muscle cell suspension is firstly injected into the assembly, as described for the other cell types, to give a layer of about 200μ to 1000μ thick. After 8 to 24 hours of the process sufficient endothelial cells are injected to give a single cell layer over the cardiac muscle cells. After a further 8 to 24 hours of the process the pulse pressure is applied for 1 to 7 days. After this period, the centrifugal tube assembly is removed from the apparatus and the foam/latex insert 142 is removed. The special inserts, with the mini-heart formed between them, and still covered with tissue culture medium, is removed. FIG. 16 shows a typical application of a mini-heart produced above. In this application the heart 152 still attached to the special inserts 140 is located in a holding device 154 and has respective one-way valves 156 fitted at each end. Any suitable design of one-way valve could be used.

The mini-heart 152 is then connected in a loop with plastic tubing as shown in FIG. 16 and any air present is purged from the system. The system is pressurised to a predetermined pressure by the hydrostatic height h of the fresh medium supply above the medium level in the mini-heart holding vessel. The pressure used may typically be between 1 and 20 gm/cm². Fresh medium can be added as required by opening a waste valve 158 to remove spent medium. The mini-heart may be made to beat by means of a mini-pacemaker device 160 connected to the mini-heart.

This set up could provide a working in-vitro mini-heart for medical research.

Referring now to FIGS. 17 to 19 certain modifications to the centrifugal tube assemblies implied in the above embodiments will now be described.

FIGS. 17(a) and (b) are longitudinal sectional views through an embodiment of centrifugal tube assembly in accordance with this invention with the end caps removed and in place respectively. In this arrangement, the centrifugal tube assembly 200 is made up a suitable plastics tube 202 made from e.g. P.T.F.E. on the centre of which is mounted a soft rubber drive wheel 204. Inside the tube 202 is located a resiliently compressible tube 206 in the form of a closed cell silicone sponge. The opposite ends of the insert 206 are tapered as shown and then mate at opposite ends with complementarily tapered cell adhesion mini tubes 208. In this embodiment, the mini tube 208 is generally incompressible and the reason for the taper is to give a gradual transition between the compressible silicone sponge and the non-compressible mini tube.

The cell adhesion mini tubes 208 are made of suitable material such that the cells deposited in the process tend to adhere thereto. The provision of the cell attachment zones at opposite ends of the inwardly facing tubular tissue formation surface helps prevent contraction of the tubular tissue structure along its axis during the maturation period. Each end of the formed tubular tissue structure is therefore fixed by cell adhesion to the surface of the mini tubes 208 and so the tubular tissue structure is held firmly between the two attachment zones. The optional attachment of a tubular scaffold insert (as described earlier) may further stabilise the tubular tissue structure, for both axial and radial cell contraction.

The adhesion mini tubes may be made of any suitable material for cell attachment and suitable for use in this system. The surface of the mini tube where cell attachment is to occur may be provided with numerous micro-projections or other suitably textured surface or surfaces to provide extra surface area and hence, extra cell attachment. The micro-projections may be selected to be long enough to penetrate all the layers of deposited cells where multiple layers are deposited.

A further modification of the above embodiments involves the sealing mechanism for the stainless steel injection tubes 218 which connect to either end of the centrifugal tube assembly 200.

FIG. 18 is a section view through one end cap assembly.

Referring to FIG. 18, the end cap assembly 210 is made up of an end cap 212 which has a portion 213 which plugs into the end of the tube 202 and is provided with twin spaced ‘O’ ring seals 214 to provide good sealing against the inner surface of the tube. The other end of the end cap 212 is received in a bearing 216. A stainless steel tube 218 remains relatively stationary as the end cap 212 rotates in use. The stainless steel tube passes centrally into the end cap 212 to align with a bore 220 in the end thereof. The stainless steel tube is sealingly located relative to the end cap 212 by means of twin-spaced O-rings 222 held apart by an ‘O’ ring spacer 224. The assembly of the ‘O’ rings 222 and the spacer 224 is held in a bore in the cap by means of a locking screw 226. The stainless steel tube passes through a stainless steel tube holder 228 and is clamped therein by means of a grub screw 230. On exiting the stainless steel tube holder 228, the stainless steel tube 218 is attached to a shut off tap 232. In order to reduce the drag or friction between the stainless steel tube 218 and the end cap which rotates relative thereto, the stainless steel tube 218 is in this example coated in a thin layer of P.T.F.E. or Teflon® to reduce friction against the ‘O’ rings 222. The ‘O’ rings 222 can be of any suitable material and could also be coated with friction reducing substances which are readily commercially available, thereby prolonging their life.

FIG. 19 shows an alternative seal arrangement intended to replace the ‘O’ rings 222 and the ‘O’ ring spacer 224 of the arrangement of FIG. 18. A unitary component 234 made of any suitable material is provided with seal lips 236 which sealingly engage the external surface of the stainless steel tube 218 and which incline towards the interior of the tube 202.

In other arrangements, instead of there being a taper at their interface, the cell adhesion tube 208 and the compressible tube 206 may have a plane butt joint.

The apparatus described in this specification may be used for laboratory or experimental purposes but equally there is a need for apparatus which may be used for the commercial preparation on a larger scale of a standard product. Described below is therefore an embodiment for the commercial preparation of Tissue Engineered Blood Vessels (T.E.B.V.), other tubular constructs (e.g. ureters) and sheets of cells such as cardiomyocytes. The production of multi-layered sheets of cells containing keratinocytes, melanocytes and fibroblasts could also be prepared for use as skin grafts. The embodiment of apparatus described below is intended for the commercial preparation of small diameter tissue engineered blood vessels. However the preparation of other tubular constructs or cellular sheets would only require modification to the proportions of the inner tubular tissue formation surface. In the embodiment described below in relation to FIGS. 20 to 24, six centrifugal tube assemblies are mounted around a common driveshaft to provide a hexagonal module or repeat unit.

The basic concept and method, follow that described above although in this case large numbers of tubular tissue structures can be prepared simultaneously in a single batch. To allow this, the arrangement described below has been modified.

The centrifugal tube assemblies 200 used in this embodiment are of the type shown in FIGS. 17 (a) and (b), with either a tapered interface or a butt interface between the cell adhesion mini tube 208 and the compressible tube 206. This embodiment is based on a hexagonal layout containing six centrifugal tube assemblies 200. The six centrifugal tube assemblies 200 are held between two hexagonal support plates 240 which are spaced apart by six hexagon spacers 242 and clamped by nuts 244. Each hexagonal support plate 240 includes six apertures for receiving respective centrifugal assemblies 200. A central aperture 246 in each hexagonal support plate rotatably supports by means of two bearings 248 a driveshaft 250 with a splined end 252. Secured near the mid-point of the driveshaft 250 is a driveshaft wheel 254 with a soft rubber external drive surface. The driveshaft is rotatably located by means of driveshaft locaters 256. As can be seen from FIG. 22, when assembled, the driveshaft wheel 254 is in driving contact with the soft rubber drive wheels 204 on each of the centrifugal tube assemblies 200. Rotation of the driveshaft 250 therefore rotates each of the centrifugal tube assemblies 200 at the same speed but in the opposite sense. The diameter of the drive wheels is chosen such that there is sufficient contact between the driveshaft wheel 254 and the drive wheels 204 on the outside of the centrifugal tube assemblies 200 to rotate without loss of traction, but without causing undue pressure on the centrifugal tube assemblies. It has been found that minimum contact is required to overcome the small amount of rotational friction from the bearings 216 and the contact between the stainless steel tube 218 and the ‘O’ rings 222, or alternative seal arrangements. As can be seen from FIGS. 24 (a) and (b), a spider fluid distribution system is set up with tubes 258 radiating out from a central tube 260 and connecting at their other ends to the taps 232 on the end caps. Thus the length of the tube connecting each centrifugal tube assembly 200 to the common tube 260 is the same. The internal diameter of the common tube 260 should be about 2.5 times that of each of the tubes 258 connecting to individual centrifugal tube assemblies, to ensure smooth flow rates.

In order for a larger number of centrifugal tube assemblies to be used at the same time for the commercial raising of tissue cultures, the hexagonal unit illustrated in FIG. 24 may be assembled in modular fashion with other like units in a frame. There are many different arrangements for combining the hexagonal repeat units and we describe below combinations of seven, twenty one, and forty nine hexagonal repeat units which would provide forty two, one hundred and twenty six and two hundred and ninety four centrifugal tube assemblies respectively.

Referring now to FIGS. 25 to 32, a frame 262 is provided to hold seven hexagonal repeat units in a hexagonal array as shown in FIG. 25 (b). The hexagon spacer bars 242 described previously are long enough to fit through the holes 264 in the frame 262 and the frame 262 can then be held in position by threaded nuts on the end of the stainless steel spacer bars. The array of seven hexagonal repeat units is now held firmly in position by each of the two end frames 262 as shown in FIGS. 26 (a) and (b). In order to supply drive to driveshaft 250 of the hexagonal repeat unit a complementary shaped drive system is provided. Referring to FIGS. 28 (a) and (b) and FIG. 29, this is done by providing a frame with space plates 264 held together in space fashion by spacer bolts 266. The drive system is provided with a central driveshaft 268 which distributes drive at the same rotational speed to seven splined drive connectors 270 which connect to splined driveshafts 250 of each hexagonal repeat unit. The drive is distributed by meshing cogs 272 of equal size. It is to be noted that adjacent outer cog pairs are alternated in the left and right position to prevent contact. This transmission is connected to the frame 262 by means of suitable extension bars.

Referring now to FIGS. 30 (a) and (b), fluid is distributed to each of the hexagonal repeat units by means of a similar distribution system (but on a larger scale to that of each individual system). A common tube 280 is connected to branches 282 in spider fashion and distributes fluid along each of these radially directed arms. The internal diameter of the common tube 280 is about 2.5 times that of the radial arms 282.

Referring now to FIGS. 31 (a) and (b) the assembly shown in FIG. 29 is pivotally mounted on a base frame 290 for movement between the position shown in FIG. 31 (a) in which the centrifugal tube assemblies 200 are horizontal, and the vertical arrangement of FIG. 31 (b) in which the centrifugal tube assemblies are vertical. This allows the assembly to be pivoted through 90° to allow the cells to be injected into the centrifugal tube assemblies when they are in the vertical position and then to be returned to the horizontal position when required for rotation and maturation.

Referring to FIG. 32, the whole assembly is then fitted inside an insulated container 292 along with a medium supply system 294, a peristaltic pump 296, a heating system 298, a programme-timed stop valve 300, a pressure monitor 302, a waste medium container 304, a cooling system 306, lines 308, 310 for the admission or withdrawal of 5% CO₂ from the medium supply 294, a high flow rate inline filter 312, a pulse pressure generator 314 and 3-way valves 316. It is preferred for all motors and pumps etc. to be of low voltage type or suitable for use in a damp environment in case of leakage or spills. A data acquisition system may be used to monitor all the relevant functions such as temperature, motor speed, flow rates, pressures etc.

The basic process of cultivation of tubular tissue structure would be similar to that described above. Pulse pressure is produced by opening and closing the solenoid stop valve for varying amounts of time and this, coupled with the flow from the peristaltic pump (with or without the auxiliary pulse pressure generator 314) is designed to be sufficient to produce the variable pulses required.

After the required time for formation of the tubular structure, the system is cooled before dismantling and removal of the individual centrifugal tubular assemblies 200. These will then be stored at about 1° C. before despatch to the recipients by overnight delivery (still maintained at 1° C.). When required for use, the centrifugal tube assembly would be opened and the tubular tissue structure (e.g. Tubular Engineered Blood Vessel) removed for use.

Quality control of the tubular tissue structure may be checked at various times throughout the process by the removal of single centrifugal tubular assemblies. This may be achieved by placing the required number of modified hexagonal repeat units at the periphery of the grouping. The apparatus may be modified so that one centrifugal tubular assembly in each of these units can be removed without dismantling or stopping the machine. This may be achieved by means of a simple slot device that allows the complete single centrifugal tubular assembly with its bearing etc. to be removed from the hexagonal repeat unit generally as described in later embodiments. After removal, the tubular tissue structure may be checked for various functions such as e.g. relaxation, contraction, burst pressure, cell and matrix content etc.

An important advantage of the arrangement described herein is that, from initial formation to final use, the engineered tubular tissue structure would remain untouched inside a controlled environment which provides protection and nourishment until the tissue engineered structure is required.

Referring now to FIGS. 33 to 35, it will be appreciated that the above embodiment of FIGS. 25 to 30 can itself be assembled with other like units to provide greater numbers of centrifugal tube assemblies. In the arrangement of FIGS. 33 to 35, three clusters of seven hexagonal repeat units are assembled together with all the centrifugal tube assemblies parallel to each other to provide an arrangement of one hundred and twenty six centrifugal tube assemblies. The drive to each hexagonal repeat unit is provided by the distributed gear arrangement of FIGS. 35 (a) and (b).

Because of the regular shape of a cluster of seven hexagonal repeat units, the array may be increased as required and FIGS. 36 and 37 show an arrangement comprising forty two hexagonal repeat units to provide a total of two hundred and ninety four centrifugal tube assemblies.

Referring now to FIGS. 38 to 46, in some instances it is desirable to have a machine which holds multiple centrifugal tube assemblies 200 but fewer than the six of the hexagonal repeat unit, particularly for small scale lab use. In addition, for lab use, it is desirable to be able to remove individual centrifugal tube assemblies without disturbing the drive to the other assemblies in the unit.

The arrangement shown in FIGS. 38 to 46 allows up to 3 centrifugal tube assemblies to be run at the same time.

The machine illustrated in these Figures also allows the use of different sized centrifugal tube assemblies at the same time; the centrifugal tube assemblies may be of different length and diameter. However the speed of rotation of each centrifugal tube assembly must be the same at any one time, because they are all driven from a common diameter drive wheel. In this arrangement the flow of medium through each centrifugal tube assembly can be on or off (by unclamping/clamping the tubes which supply medium to the centrifugal tube assembly). If the flow is on, then the flow of medium through each centrifugal tube assembly will normally be at the same rate. Each centrifugal tube assembly has its own stop valve and the pulse pressure to each assembly can be individually varied for the same tissue culture medium flow rate, simply by entering the length of time for which the stop valve for each centrifugal tube assembly is open or closed.

The machine is designed to be stand alone without requiring apparatus such as incubators etc. A supply of 5% CO₂ can be provided for tissue culture medium which requires this gas mixture to maintain its buffering capacity. All motors, heaters and fans inside the operational part of the machine may conveniently be powered by a low DC voltage of 12 or 24 volts, so that the risk of electric shock or electrocution due to liquid spillages etc. is minimal. The low DC voltage may be supplied from a separate control box by a mains voltage operated power pack.

FIGS. 38 (a) to (d) are plan views on the support plates 320, 322, 324 and 326, which form the main support of the machine. In this particular embodiment the support plates may start off as four identical square plates of, in this example, 160 mm² and about 15-20 mm thick. They may be made out of any suitable material including stainless steel or high quality engineering plastic. Eight 10 mm holes and one 21 mm hole 330 are drilled in each of the four plates, and the 21 mm hole in the fourth plate 326 is enlarged to a 50 mm hole 332. Three rectangular slots 334 are then cut in three sides of the first two plates 320 and 322 and further holes (not shown) are drilled in the edges of these two plates for later fitting of spring/plungers and adjustment plates. The second plate is then cut into four portions and the bottom portion discarded.

Referring now to FIG. 39, the plates 320, 322, 324 and 326, are fitted together with connecting rods 336 which in this example are of stainless steel, approximately 400 mm long and 10 mm in diameter. The first, third and fourth plates 320, 324 and 326 are bolted to a base plate 338 by bolts 340, a driveshaft 342 with a drive wheel 344 attached passes centrally through the aligned apertures 330 and 332 by means of bearings 344. The driveshaft 342 is located by tubular screw clamps 346 and a motor 348 connected to the fourth mounting plate 326 by bolts and connected to the driveshaft 342 by means of an anti-vibration connector 350. The three divided quarter plates 322 can slide freely, and independently, along the stainless steel rods between the first and third blocks 320 and 324. The stainless steel rods are fixed in the first and fourth plates 320 and 326.

Referring now to FIGS. 40 and 41, each centrifugal tube assembly 200 is fitted at its ends with a sliding holder 352 which has a central locating hole 354 and is fashioned to provide two lateral flanges 356. The sliding holders 352 are arranged to be fitted to each end of the centrifugal tube assembly by suitable bearings. Each centrifugal tube assembly, with a sliding holder 352 at opposite ends is located at one end in the rectangular recess 334 of the first support plate 320 and at its other end in the rectangular recess 334 of one of the divided quarter plates 322. The width of the main body portion of the sliding holder 352 is dimensioned to be a sliding fit in the recess, with the flanges 356 engaging the internal sides of the support plates 320, 322 relative to the centrifugal tube assembly 200, to provide longitudinal location but allowing rotation of the centrifugal tube assembly 200.

Referring now to FIGS. 42 (a) to (d) these show detailed views on the inter-engagement of the centrifugal tube assembly 200, its sliding holder 352 and the quarter block 322. It will be seen that each recess 334 in the plate is provided in its base with a push rod 360 biased away from the base by a spring 362. The assembly is such that the push rod 360 bears on a surface of the sliding holder 352 to urge it in a direction out of the recess 334. The sliding holder 352 is held in the recess by means of an adjuster plate 364 which is bolted over the recess 334 by bolts 366. At its centre, each adjustment plate 364 has a spring-loaded bolt assembly 368 which can be used to adjust the height of the sliding holder 352.

Referring now to FIG. 43, this shows an assembled arrangement with 3 centrifugal tube assemblies 200 each with drive wheels 204 mounted in the assembly. Each centrifugal tube assembly 200 is fitted between the first plate 320 and its respective slideable quarter plate 322 which is adjusted so that there is no free-play of the sliding holders 352 in the longitudinal direction between these plates. Locking clamps 370 are slid along the connecting rods 336 to abut the quarter plate 322 and then tightened to maintain the position.

The springs in the spring-loaded bolt assemblies 368 are slightly stronger than the springs 362 in the base of the recess, so that when the spring bolt assemblies at each end of a given centrifugal tube assembly 200 are evenly tightened, the drive wheel 204 is kept pushed against the driveshaft wheel 344 by positive spring pressure (see FIG. 44 (b)). Only a small amount of positive spring pressure is required to provide the small amount of friction required to rotate the centrifugal tube assemblies 200.

Referring now to FIG. 45, the complete mechanism of FIG. 6, fixed to the base plate 338 is located within an insulated box 370 with the fourth plate 326 outside the insulated box and the connecting rods 342 passing through complementary holes in the end wall of the box. The box contains several other active/control components including a PTC (positive temperature coefficient thermistor) controlled heater/radiator/fan 372, a medium supply 374 and a waste medium container 376 each in swing containers to allow them to maintain their orientation when the equipment is tilted vertically (as to be described below), a peristaltic pump tube holders 378 with an associated motor and gearbox 380, solenoid stop valves 382 for modulating the pressure in each of the centrifugal tube assemblies 200 to provide a pulsed effect, a secondary fan 384, a PID (proportional-integral-derivative) temperature sensor 386, temperature detectors 388, a pressure detector (not shown) and tubing for directing the medium to and from each of the centrifugal tube assemblies 200. It will be noted that the motors 380 and 348 are both located outside the box 370.

A control box 390 is separate from the mechanical part of the machine and houses the DC power supply 392 for the motors 348 and 380, the motor speed controllers 394 for each of the motors, a power supply 396 for the cooling fans, PID heater controller 398, a pressure measuring device 400, an electronic on/off switch 402 for the solenoid stop valves 382 and digital read outs 404 for indicating the operating status of the equipment. These components are all connected to their relevant control mechanisms by respective wires. A data acquisition system 406 can be added if required. Also, the control of the various components can be effected by computerised control.

Referring now to FIGS. 46 (a) and (b) the box 370 with the removable lid 408 is pivotally attached at its base by means of a bracket 410 to a main stand 412. A gas strut 414 extends between the stand 412 and the base plate 338 to allow the entire assembly to be rotated between the position in FIG. 46 (a), in which the centrifugal tube assemblies 200 are horizontal, to the vertical tilted position shown in FIG. 46 (b) where the centrifugal tube assemblies 200 are disposed vertically. The vertical position is required for cell injection while the horizontal position is preferred for the remainder of the process. As indicated previously, the swing holders for the supply medium container 374 and the waste medium container 376 allow these containers to remain upright with the machine when it moves to its vertical position.

Variable pulse pressure may be produced in the system by a combination of modulation of the speed of the peristaltic pump 378 and the period for which the solenoid stop valves 382 are open and closed. For example, a low pump speed with a stop valve close time of 0.2 second and an open time of 0.8 second would produce 60 low pressure pulses per minute. On the other hand, a higher pump speed with a stop valve close time of 0.3 second and an open time of 0.2 second would produce 120 higher pressure pulses per minute. The pressure pulses may be measured at the medium exit from the centrifugal tube assembly by a laser micro-sensor (not shown) connected to the pressure measuring device 400. If required, an auxiliary pulsed pressure generator could be fitted into the system to allow further variation of the pulse pressure.

For significantly larger or smaller centrifugal tube assemblies 200, the relevant parts of the machine could be made from different sized components.

The sequence of cell injection, rotation speeds etc. may follow that described above, they can of course be varied to suit different protocols.

To remove a centrifugal tube assembly 200 whilst the machine is operational, the supply tube is simply disconnected from the peristaltic pump drive 378 and the valves at each end of the centrifugal tube assembly are closed. The spring-loaded bolt assemblies 368 at each end of the centrifugal tube assembly are unscrewed until the drive wheel 204 is urged clear of the drive wheel 344 on the driveshaft by the spring/push rod arrangement 362/360 and the centrifugal tube assembly 200 will then stop rotating. The securing clamps 370 are loosened so that the relevant quarter plate 322 can be slid away to release the centrifugal tube assembly from the plates 322 and 320. The relevant tubing can then be disconnected and the centrifugal tube assembly 200 disassembled to allow removal of the formed cellular structure. The portions of the machine that need to be sterile, for example the centrifugal tube assemblies, the connecting tubes, valves etc. are preferably made from materials that can be autoclaved for reuse.

Referring now to FIGS. 47 to 53, there will now be described an embodiment which effectively extends the principles described above in connection with the arrangement intended for lab use with 3 centrifugal tube assemblies 200 and to apply it to a hexagonal arrangement which is similar in configuration to that of the hexagonal repeat units described above. This particular embodiment is intended for lab based research for the preparation of tissue engineered tubular constructs containing one or more different cell types. The machine can be used with up to 6 centrifugal tube assemblies 200, but they must be of the same dimensions and so the conditions inside each centrifugal tube assembly 200 would be the same. All the centrifugal tube assemblies 200 are supplied with tissue culture medium from a common tube so that the tissue culture medium flow rate and pressure pulses are the same for each centrifugal tube assembly. This is because the solenoid stop valve and the peristaltic pump are situated on the common tube as is the cell injection site.

This arrangement allows for removal of individual centrifugal tube assemblies 200 at various times throughout the procedure, without interrupting the operation in rotation of the other centrifugal tube assemblies. The general mode of operation would be to set up the machine, inject the different cell types according to the particular injection protocol so that each centrifugal tube assembly would contain an identical arrangement of cell layers. After about 24 hours, the pressure pulses would be started and gradually increase daily over the required period of time (which will depend on the particular protocol but could typically vary from 7 to 14 days). At predetermined times throughout the experimental procedure, single centrifugal tube assemblies 200 are removed from the machine for evaluation of the tubular constructs, and the pressure pulses supplied along the common supply tube adjusted before a centrifugal tube assembly is removed, to compensate for the increased flow rate caused by removal thereof.

The machine is designed to be stand alone and does not require apparatus such as incubators etc. The supply of 5% CO₂ can be provided for tissue culture mediums that require this gas mixture to maintain buffering capacity. As previously, all motors, heaters and fans inside the operational part of the machine are powered by a low DC voltage of 12 or 24 volts so that the risk of electrocution due to liquid spillages etc is minimal. The low DC voltage may be supplied from a separate control box by mains voltage operated power packs. Referring now more specifically to FIGS. 47 to 53, the arrangement comprises two space hexagonal support blocks in place of the four plates of the arrangement just described. Each hexagonal block has a rectangular recess 502 which slideably receives a sliding holder 352 of the type previously described. Each sliding holder is releasably held in its recess by an adjustment plate 364 and bolts 366, the adjustment plate 364 has a spring-loaded bolt assembly 368 which cooperates with a spring-push rod arrangement 362/360 to allow the radial position of the sliding holder 352 to be adjusted. As previously, the enlarged flanges 356 of the sliding holders bear on the respective facing surfaces of the hexagonal blocks to locate the centrifugal tube assemblies 200.

A driveshaft 504 passes centrally through the hexagonal plates 500 and carries a drive wheel 506 which engages the drive wheels 204 on each of the centrifugal tube assemblies 200, with the assemblies 200 being urged into driving engagement with the drive wheel 506 by suitable adjustment of the spring-loaded bolt assemblies 368.

The hexagonal plates 500 may be made of any suitable material including stainless steel or high quality engineering plastics. As with the support plates for the arrangement for supporting three centrifugal tube assemblies 200 described above, six holes 510 are drilled at the corners of the hexagon to receive connecting rods, and a single central hole is drilled to receive the driveshaft. The rectangular recess 502 may be removed by milling. As previously holes are also drilled in the base of each recess and into the flat of each hexagonal side for the fitment of spring/push rods and the adjustment plates.

Six connecting rods (not shown but similar as in the previous embodiment) are fitted through the holes 510 on the hexagon plates and secured by locking nuts. It will be appreciated that the length of the centrifugal tube assemblies 200 is determined by the spacing between the plates and therefore the length of the connecting rods and so the machine can be designed to accommodate any reasonable length centrifugal tube assembly. As previously, the motor driveshaft 504 is located in the centre of each hexagon by bearings and tubular driveshaft locater clamps 508.

To assemble the machine, sliding holders 352 are fitted to each end of the centrifugal tube assembly using bearings and needle clamps fitted to the other side. The push rods/springs 360/362 are fitted into the base of the recess and each centrifugal tube assembly rotatably mounted between sliding holders 352 is placed in the rectangular cutouts between the hexagon blocks and there should be minimum axial free-play of the sliders between the hexagons. The adjustment plates 364 are bolted to retain the centrifugal tube assemblies and the spring-loaded adjustment bolts 368 tightened to urge each drive wheel 204 into contact with the driveshaft wheel 506. As previously, the springs in the bolt assemblies 368 are thus slightly stronger than those in the base of the recess so that when the spring bolts at each end of the centrifugal tube assembly are evenly tightened, the drive wheel 204 is kept pushed against the driveshaft wheel by positive spring pressure. Only a small amount of spring pressure is required to provide the small amount of friction required to rotate the centrifugal tube assembly 200. The medium supply and return tubes each comprise spider arrangements with a central common tube 510 and radiating branches 512 as seen in FIGS. 50 (a) and (b).

The complete assembly is supported by the driveshaft 504. This is achieved by two driveshaft supports 514 with bearings and driveshaft locaters fitted to a base plate 516. This arrangement allows the hexagon plates 500 to rotate on the driveshaft relative to the base plate to allow top access to each of the centrifugal tube assemblies. Rotation in this manner is limited to half a turn in either direction and a lock screw 518 extends from the right hand drive support (as viewed in FIG. 51) to lock the hexagonal assembly. The motor 520 is bolted to a motor support 522 which is also fixed to the base plate 516 and the motor is connected to the driveshaft by an anti-vibration connector 524. As in the previous arrangement, the assembly of centrifugal tube assemblies 200 is located in an insulated box 526 with a removable lid 528. The base plate 516 is pivotally attached to a stand 530 by means of a bracket 532 and a gas strut 534 extends between the base plate 516 and the stand 530. This allows the machine to be tilted between the horizontal position shown in FIG. 52 (a) and the vertical position shown in FIG. 52 (b).

As previously, the insulated box 526 contains all the components of the hexagonal assembly apart from the motor 520 and a motor 522 for the peristaltic pump. The insulated box contains a PTC heater/radiator/fan 530, medium supply and waste bottles 532 and 534 in swing holders as before, a peristaltic pump tube holder 536, a secondary fan 538, a PID temperature sensor 540, temperature detectors 542, a 12 volt solenoid stop valve 544, a pressure detector 546 and all medium tubing connectors.

A completely separate control box 548 houses 24 and 12 volt power supply 550, 552 respectively, motor speed controllers 554, 556 for the motors 520 and 522, a PID heater controller 558, a pulse pressure controlling device 560, an on/off time switch 562 for the solenoid valve 544 and digital readouts 564. All these components are connected to the relevant control mechanism by suitable wiring. A data acquisition system can be added if required, as can be computerised control over most of the system already described.

As previously, variable pulse pressure is achievable to provide different pulse rates, magnitudes, profiles, etc. by control of the on/off period of the solenoid stop valve 544 and the speed of the peristaltic pump 536. The pressure control mechanism and variations thereof described above may be used in this apparatus. The sequence of cell injection, rotation speeds etc. is similar to that already described above but can be varied to suit different protocols.

To remove a centrifugal tube assembly whilst the machine is in operation, the valves at each end of the centrifugal tube assembly to be removed are turned off. The hexagonal assembly may be rotated by releasing the rotation lock screw 518 and rotating the assembly to present the required centrifugal tube assembly uppermost. The spring-loaded bolts 368 at either end of the centrifugal tube assembly are slackened evenly until the drive wheel 204 on the centrifugal tube assembly 200 is clear of the drive wheel 506, and the centrifugal tube assembly 200 stops rotating. The centrifugal tube assembly 200 may then be released from the assembly by removing the adjustment plates and clamps etc. Cellular tubular construct can now be removed from the centrifugal tube assembly and used as required.

It will be appreciated that the hexagonal unit described and illustrated in relation to FIGS. 17 to 24 could be used for the commercial preparation of tubular constructs. The hexagonal unit as described earlier, with fixed position centrifugal tube assemblies 200 would be fitted inside the machine instead of the just described version with individually removable centrifugal tube assemblies. This however would mean that all the centrifugal tube assemblies would have to be harvested at the same time and therefore produce six identical tubular constructs.

1-37. (canceled)

38. A method of producing a cellular tissue structure comprising a wall surrounding a space or passage, which method comprises the steps of:—
(i) introducing into a vessel at least one suspension of cells, said vessel having therein an internal tissue formation surface;
(ii) rotating said vessel about a rotary axis to cause the cells in said suspension to move under the influence of centrifugal force to collect and form a layer on said tissue formation surface, and
(iii) exposing said layer to a tissue culture medium by passing said tissue culture medium through said vessel.

39. A method according to claim 38, wherein a first cell suspension containing cells of a first type is introduced into said vessel and, following step (iii), a second cell suspension containing cells of a second type is introduced into said vessel and steps (ii) and (iii) are repeated with said second cell suspension.

40. A method according to claim 39, wherein a third cell suspension containing cells of a third type is introduced into said vessel and steps (ii) and (iii) are repeated with said third cell suspension.

41. A method according to claim 38, wherein said at least one cell suspension comprises a suspension comprising a plurality of different cell types.

42. A method according to claim 38 which includes applying a time-varying pressure to said layer.

43. A method according to claim 38, wherein said tissue formation surface is provided on the inner surface of a generally tubular tissue formation insert element which fits within said vessel.

44. A method according to claim 43, wherein said insert element is made of a resiliently deformable material such that the inner radial dimension thereof varies in response to said time-varying pressure.

45. A method according to claim 44, wherein said insert element is made of a closed cell foam material.

46. A method according to claim 43, wherein a further tubular insert element of support material is provided within said tissue formation insert.

47. A method according to claim 38, which includes providing a tissue scaffolding structure in said vessel and associated with said tissue formation layer.

48. A method according to claim 38, wherein each cell suspension is caused or allowed to become generally uniform after introduction into said vessel by rotating said vessel, during or immediately after step (i).

49. A method according to claim 48, wherein said vessel is rotated at a speed of between 5 and 15 rpm to cause the cell suspension to become generally uniform.

50. A method according to claim 38, wherein said vessel is rotated at a speed of between 500 and 3000 rpm at least during an initial phase of step (ii).

51. A method according to claim 50, wherein said vessel is rotated at a speed between 1500 and 2500 rpm during an initial phase, with the speed thereafter being reduced to between 500 and 1500 rpm.

52. A method according to claim 38, wherein step (ii) is continued for a period of between 5 and 24 hours.

53. A method according to claim 38, wherein said time-varying pressure comprises a pulsed pressure cycle.

54. A method according to claim 53, wherein at least one of the frequency and magnitude of the pulses increases with time.

55. A method according to claim 54, wherein the magnitude the pulses lies in the range from 1 gm/cm to 200 gm/cm@, and the frequency thereof lies in the range of from 10 pulses/minute to 80 pulses/minute.

56. Apparatus for producing a cellular tissue structure comprising a wall surrounding a space or passage, said apparatus comprising:—an elongate culture vessel having therein an internal tissue formation surface; cell suspension supply means for introducing into said vessel at least one cell suspension; mounting means for supporting said vessel for rotation about a rotary axis; rotary drive means for rotating said vessel; and pressure modulation means for varying the pressure within said vessel.

57. Apparatus according to claim 56, wherein said tissue formation surface comprises a radially inner surface of a tissue formation insert element removably located in said vessel.

58. Apparatus according to claim 57, wherein said tissue formation insert element has an inner surface of elongate, generally tubular form.

59. Apparatus according to claim 58, wherein said tissue formation element is made of a resiliently deformable material.

60. Apparatus according to claim 59, wherein said tissue formation element is made of a closed cell foam material.

61. Apparatus according to claim 58, which includes a further insert element of support material located within said tissue formation insert element.

62. Apparatus according to claim 56, wherein a tissue scaffolding structure is associated with said tissue formation surface.

63. Apparatus according to claim 56, wherein said vessel comprises a hollow cylindrical tube and respective end caps sealingly connected to opposite ends of said tube, each end cap including a flow passage communicating with the interior of said tube.

64. Apparatus according to claim 63, wherein at least one of said end caps is removable.

65. Apparatus according to claim 62, wherein each said flow passage is defined by a hollow tube removably connected to the associated end cap.

66. Apparatus for culturing cells, which apparatus comprises:—a plurality of generally elongate culture vessels mounted on a support means with their longitudinally axis generally parallel and each mounted for rotation about its respective longitudinal axis, and common drive means for rotating each of said culture vessels.

67. Apparatus according to claim 66, wherein said common drive means includes a drive element for engaging the circumference of each of said culture vessels to impart drive thereto.

68. Apparatus according to claim 67, wherein said culture vessels are disposed radially around said drive element.

69. Apparatus according to claim 66, wherein said support means is configured to allow removal of one of said culture vessels without disturbing the drive to the other culture vessels.

70. Apparatus according to claim 69, wherein said support means comprises means for transversely slideably receiving opposite end regions of said culture vessels and means for releasable locking said culture vessels to said support means.

71. Apparatus according to claim 69, wherein said support means includes end clamp means for clamping opposite end region of culture vessel, at least one of said clamp means being moveable longitudinally between engaging and disengaged positions, and means for releasably locking said clamp means.

72. Apparatus according to claim 38, which includes a plurality of modules each comprising a plurality of culture vessels rotatably mounted on support means and rotatably driven by a module common drive means, with the modules being interconnected to allow transmission of drive between the modules.

73. An engineered tissue structure produced in accordance with the method of claim 38.

74. An elongate culture vessel for use in the apparatus as claimed in claim 56.
